(19) United States
(12) Patent Application Publication
(10) Pub. No.: US 2010/0222752 A1
(43) Pub. Date: Sep. 2, 2010

(54) OPHTHALMIC FLUID DELIVERY SYSTEM

(76) Inventors: James F. Collins, JR., Long Island City, NY (US); Michael J. Jobin, Boston, MA (US)

Correspondence Address: BRINKS HOFER GILSON & LIONE, P.O. BOX 10395, CHICAGO, IL, 60610, US

(21) Appl. No.: 12/650,008

(22) Filed: Dec. 30, 2009

Related U.S. Application Data
(63) Continuation-in-part of application No. 10/851,611, filed on May 20, 2004.
(60) Provisional application No. 61/203,908, filed on Dec. 30, 2008.
(60) Provisional application No. 60/471,883, filed on May 20, 2003.
(60) Provisional application No. 60/485,305, filed on Jul. 3, 2003.

(51) Int. Cl. A61F 9/00

(52) U.S. Cl. 604/296

(57) ABSTRACT

An ophthalmic fluid atomizer including a body having a proximal end and a distal end and a container connected to the body, wherein the container contains an ophthalmic fluid disposed therein. The atomizer further including a discharge plate disposed at the distal end and a wick extending from the container to the discharge plate and transmitting the ophthalmic fluid from the container to the discharge plate via capillary action. Transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye. The atomizer further includes a processor that controls vibration of the discharge plate which causes an aerosol mist to form and an activation switch operatively coupled to the processor to activate the transmission of the ophthalmic fluid from the discharge plate.

### CROSS REFERENCE TO RELATED APPLICATIONS

The present application claims priority under 35 U.S.C. §119(e) to U.S. Provisional Application No. 61/203,908, filed on Dec. 30, 2008 and the present application is a continuation-in-part of U.S. patent application Ser. No. 10/851,611, filed on May 20, 2004, presently pending, which claims priority under 35 U.S.C. §119(e) to both U.S. Provisional Application No. 60/485,305, filed on Jul. 3, 2003 and U.S. Provisional Application No. 60/471,883, filed on May 20, 2003, wherein each of the above mentioned U.S. Patent and U.S. Provisional Applications are incorporated herein by reference.

### FIELD OF THE INVENTION

The present invention relates to drug delivery devices for dispensing liquid as an aerosol or atomized mist and, more particularly, for dispensing medicaments or ophthalmic fluids to the eye.

### BACKGROUND

Presently, conventional eye drops are the standard means of delivering medicaments to the eye. This means of ophthalmic drug delivery, however, has numerous problems. For example, the average eye drop (approximately 50 micro liters) far exceeds the eye's capacity (7 micro liters in the pre-corneal tear film and a maximum of about 30 micro liters in the lower cul-de-sac) effectively destabilizing and stripping the natural tear film. This results in a brief period of massive over-dosage, which is quickly cleared by reflex lacrimation, blinking and nasolacrimal drainage, resulting in sub-therapeutic drug levels until the next medication application. This approach represents very inefficient pharmacokinetics. Far smaller volumes of medicament (approximately one tenth of a conventional drop) are desirable and are, in fact, retained by the eye and “bio-available” for a substantially longer time.

Attempts to prolong ocular contact time by various adaptations, such as the use of particulate suspensions, have led to other drawbacks including ocular irritation and excessively slow drug release. Ointments and gels, though providing prolonged contact time, create obvious visual disturbances.

Further, local irritations and toxicities often result from the regular use of eye drops. These situations vary widely depending on the pharmacologic agent, preservatives and other additives being used, but this is clearly a very non-physiologic and inefficient system of medication administration. Chronic use of eye drops for such conditions as glaucoma and prolonged infections and inflammations can, in fact, cause substantial morbidity. Additionally, serious and even fatal reactions to sympathomimetic and beta-adrenergic blocking agents have occurred as a result of systemic absorption of eye drops via nasolacrimal drainage.

Besides the above issues, there are a great many difficulties that patients experience with the mechanics of eye drop administration. Elderly patients, the largest group of eye drop users, often have hand-eye coordination problems, tremors or arthritis, affecting the hands and/or the cervical spine, making eye drop administration difficult if not impossible. Many users report that they have trouble keeping track of their regimens and often repeat doses or miss them entirely, suffering potential consequences in either event. Further, pediatric patients, often unable to comprehend the reasons and benefits behind the administration of eye medication, often fight such application, typically resulting in underdosing due to the patient's attempts to prevent the eye drops from being administered, or overdosing, as a result of the administrator's attempt to ensure that sufficient dosage is being applied.

Additionally, very few regular users of eye drops, in any age group, actually observe the ideal technique of administration, including tear sac compression, to minimize excretory loss and potential systemic absorption. It is sometimes difficult to tell if the drop was properly instilled. Direct application to the cornea can result in the drop “bouncing” from the eye with little or no benefit.

Regular eye drop users commonly report using several drops which “missed” the eye until they are sure they properly instilled the drop. Also, many eye drop bottles are fabricated in such a way that loss is unavoidable as soon as the dropper is tilted. Finally, a significant number of regular users put another drop or two in the eye “just to be sure.” All of the above represent needless waste of expensive medication (many glaucoma medications cost $70-$80 for a 5 ml bottle) and also increased the risk of side effects, while actually reducing the therapeutic benefit.

The ophthalmic literature is rife with references to the need for a better means of ophthalmic drug delivery. With an estimate of 25 million users of eye drops in the United States alone, the magnitude of the public health issue is considerable. Accordingly, a new means of ophthalmic drug delivery is needed.

The concept of “spraying” medicated solutions on to the eye is not a new one. A number of devices have been conceptualized and developed for this purpose. Various means of atomizing and propelling solutions including mechanical pumps, gas-propelled jets and pistons, etc. Which have inherent drawbacks relating to difficulties with calibrating the flow velocity, volume and particle size of the emitted spray. See, for example, U.S. Pat. Nos. 3,170,462; 5,630,793; and 6,062,212.

It is hypothesized that the generated mist will expand and “therapeutically alter” but not significantly disrupt the physiologic tear film allowing for a more natural process in the transmission of therapeutic agents to the surface and the interior of the eye. A much smaller volume of solution can be administered below the blink and lacrimation thresholds, allowing for a prolonged time of application. The aggregate administration of a drug in thousands of 5-micron particles should significantly exceed that of a single eye drop, leading to greater concentrations of the drug (bioavailability). Furthermore, the surface tension of a standard drop is a barrier to “mixing” and tear film incorporation. This problem is expected to be avoided with micronebulization.

An additional benefit to mist administration of eye medications is the avoidance of dropper bottle contamination which commonly occurs from contact with the eyelid. In the professional office setting, this problem has led to many documented epidemics of viral keratoconjunctivitis. During medication administration via a dropper bottle to a patient with viral keratoconjunctivitis, the bottle tip may inadvertently touch the eye or eyelid of the affected patient, transferring the virus to the bottle tip. Subsequent medication administrations to other patients using the same dropper bottle transmits the virus to those patients.

Some of the beneficial features of an ophthalmic medication spray dispenser include the following: great ease of use; can be used in any “attitude” (ie., With patient sitting, erect, lying down, head tilted back, etc.); abbreviated treatment cycle as compared to eye drop usage; improved bioavailability/efficacy; improved safety (reduced local and systemic side effects); improved sterility; increased compliance due to ease of use and “alert” systems; possibility of singular efficacy in the treatment of certain vision threatening infections; conservation of material (reduced volume, diminished waste/loss); and system (fixation target to help ensure proper application).

It would be beneficial to provide a system for applying the desired small amounts (7 to 10 micro liters) of optical medication, along with at least some of the above-listed beneficial features, while eliminating the drawbacks associated with previous means of drug delivery.

### BRIEF SUMMARY

One aspect of the present invention regards an ophthalmic fluid atomizer configured to safely deliver an ophthalmic fluid to an eye, the ophthalmic fluid atomizer including a body having a proximal end and a distal end and a container connected to the body, wherein the container contains an ophthalmic fluid disposed therein. The atomizer further including a discharge plate disposed at the distal end, wherein the discharge plate includes a plurality of openings extending therethrough and a wick extending from the container to the discharge plate and transmitting the ophthalmic fluid from the container to the discharge plate via capillary action. Transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye. The atomizer further includes a processor that controls vibration of the discharge plate which causes an aerosol mist to form and an activation switch operatively coupled to the processor to activate the processor.

A second aspect of the present invention regards an ophthalmic fluid atomizer configured to safely deliver an ophthalmic fluid to an eye, the ophthalmic fluid atomizer including a body having a proximal end and a distal end and a medule system releasably connected to the body. The medule system includes a container containing an ophthalmic fluid disposed therein and defining a first opening and a wick that is inserted into the opening and extending into the container so as to contact the ophthalmic fluid. The atomizer includes a discharge plate disposed at the distal end, wherein the discharge plate includes a plurality of openings extending therethrough, wherein the wick extends from the container to the discharge plate and transmits the ophthalmic fluid from the container to the discharge plate via capillary action, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye.

A third aspect of the present invention regards a medule system that includes a container containing an ophthalmic fluid disposed therein and defining a first opening and a plug inserted into the first opening, the plug defining a second opening. The medule system further includes a wick that is inserted into the first opening and the second opening and extending into the container so as to contact the ophthalmic fluid.

A fourth aspect of the present invention regards an ophthalmic fluid atomizer configured to safely deliver an ophthalmic fluid to an eye, the ophthalmic fluid atomizer including a body having a proximal end and a distal end and a container connected to the body, wherein the container contains an ophthalmic fluid disposed therein. The atomizer including a discharge plate disposed at the distal end, wherein the discharge plate includes a plurality of openings extending therethrough and a prime mover extending from the container to the discharge plate and transmitting the ophthalmic fluid from the container to the discharge plate. The transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye. The atomizer further including a glowing alignment device surrounding the prime mover, a processor that controls vibration of the discharge plate which causes an aerosol mist to form and an activation switch operatively coupled to the processor to activate the processor.

A fifth aspect of the present invention regards an ophthalmic fluid atomizer configured to safely deliver an ophthalmic fluid to an eye, the ophthalmic fluid atomizer including a body having a proximal end and a distal end and a container connected to the body, wherein the container contains an ophthalmic fluid disposed therein and the container comprises indicia indicative of the identity of the ophthalmic fluid. The atomizer including a discharge plate disposed at the distal end, wherein the discharge plate includes a plurality of openings extending therethrough and a prime mover extending from the container to the discharge plate and transmitting the ophthalmic fluid from the container to the discharge plate. Transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye. The atomizer further including a cap that covers the discharge plate and the prime mover, wherein a portion of the cap is transparent and positioned so that a user can see the indicia through the portion of the cap.

A sixth aspect of the present invention regards an ophthalmic fluid atomizer configured to safely deliver an ophthalmic fluid to an eye, the ophthalmic fluid atomizer including a body having a proximal end and a distal end and a container connected to the body, wherein the container contains an ophthalmic fluid disposed therein. The atomizer further including a discharge plate disposed at the distal end, wherein the discharge plate includes a plurality of openings extending therethrough and a prime mover extending from the container to the discharge plate and transmitting the ophthalmic fluid from the container to the discharge plate. Transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye. The atomizer further includes a closure element that moves from a first position to a second position, wherein the closure element at the first position prevents the plume from reaching the eye and the closure element at the second position allows the plume to reach the eye.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain features of the invention. In the drawings:

FIG. 1 is a side elevational view, partially broken away, of a mist spraying device according to a first embodiment of the present invention.

FIG. 2 is an exploded view of the device of FIG. 1.

FIG. 3 is an enlarged side profile view of a first embodiment of a fluid reservoir connected to the device.

FIG. 4 is a side profile view showing the device being used to spray a mist into a patient's eye.

FIG. 5 is a side profile view of the first embodiment of the fluid reservoir shown in FIG. 3, having been removed from the device.

FIG. 6 is an enlarged side profile view of a second embodiment of a fluid reservoir.

FIG. 7 is an enlarged side profile view of a third embodiment of a fluid reservoir.

FIG. 8 is a perspective view of the reservoir of FIG. 7.

FIG. 9 is an enlarged side view, in section, of a prime mover inserted into the device.

FIG. 10 is an enlarged exploded perspective view of a nozzle assembly of the device.

FIG. 11 is an enlarged side view, in section, of the nozzle assembly of the device.

FIG. 12a is an enlarged partial sectional view of a first embodiment of the mesh plate of the nozzle assembly.

FIG. 12b is an enlarged partial sectional view of a second embodiment of the mesh plate of the nozzle assembly.

FIG. 12c is an enlarged partial sectional view of a third embodiment of the mesh plate of the nozzle assembly.

FIG. 12d is an enlarged partial sectional view of a fourth embodiment of the mesh plate of the nozzle assembly.

FIG. 13a is a top plan view of a first embodiment of a mesh plate.

FIG. 13b is a top plan view of a second embodiment of a mesh plate.

FIG. 13c is a side view, in section of a third embodiment of a mesh plate.

FIG. 13d is a side view, in section, of a fourth embodiment of a mesh plate.

FIG. 13e is an enlarged partial sectional view of a fifth embodiment of a mesh plate.

FIG. 14 is a perspective view of the device showing an optional dosage adjustment feature.

FIG. 15a is a perspective view of the device showing a first embodiment of the dosage adjustment feature.

FIG. 15b is a perspective view of the device showing a second embodiment of the dosage adjustment feature.

FIG. 15c is a perspective view of the device showing a third embodiment of the dosage adjustment feature.

FIG. 16 is a top plan view showing the targeting device of FIG. 14.

FIG. 17a is a schematic view of a first embodiment of a targeting mechanism showing the device too close to the target.

FIG. 17b is a schematic view of the first embodiment of the targeting mechanism showing the device a correct distance from the target.

FIG. 17c is a schematic view of the first embodiment of the targeting mechanism showing the device too far from the target.

FIG. 18a is a schematic view of a second embodiment of a targeting mechanism showing the device too close to the target.

FIG. 18b is a schematic view of the second embodiment of the targeting mechanism showing the device a correct distance from the target.

FIG. 18c is a schematic view of the second embodiment of the targeting mechanism showing the device too far from the target.

FIG. 19a is a schematic view of a third embodiment of a targeting mechanism showing the device too close to the target.

FIG. 19b is a schematic view of the third embodiment of the targeting mechanism showing the device a correct distance from the target.

FIG. 19c is a schematic view of the third embodiment of the targeting mechanism showing the device too far from the target.

FIG. 20a is a schematic view of a fourth embodiment of a targeting mechanism showing the device too close to the target.

FIG. 20b is a schematic view of the fourth embodiment of the targeting mechanism showing the device a correct distance from the target.

FIG. 20c is a schematic view of the fourth embodiment of the targeting mechanism showing the device too far from the target.

FIG. 21a is a schematic view of a fifth embodiment of a targeting mechanism showing the device too close to the target.

FIG. 21b is a schematic view of the fifth embodiment of the targeting mechanism showing the device a correct distance from the target.

FIG. 21c is a schematic view of the fifth embodiment of the targeting mechanism showing the device too far from the target.

FIG. 22a is a side elevational view of a mechanical targeting device according to the present invention.

FIG. 22b is a top plan view of a proximal end of the mechanical targeting device shown in FIG. 22a, being used on a patient.

FIG. 23 is a schematic view of an electronic control system for the device.

FIG. 24 is a perspective view of an alternative embodiment of the device according to the present invention.

FIG. 25 is a perspective view of another alternative embodiment of the device according to the present invention.

FIG. 26 is a perspective view showing self-administration of medication using the device.

FIG. 27 is a perspective view showing administration of medication by one person to another using the device.

FIG. 28 is a front perspective view of another embodiment of a mist spraying device according to the present invention.

FIG. 29 is a left side perspective view of the mist spraying device of FIG. 28.

FIG. 30 is a partially exposed view of the mist spraying device of FIG. 28 with wiring in place.

FIG. 31 is a schematic view of the mist spraying device of FIG. 28 wherein the wiring of FIG. 30 is removed.

FIG. 32 is a schematic and cross-sectional view of the mist spraying device of FIG. 28 with wiring removed.

FIG. 33 is a perspective view of a core portion of a medule and nozzle assembly to be used with the mist spraying device of FIG. 28.

FIG. 34 is a cross-sectional view of the core portion of the medule and nozzle assembly of FIG. 33.

FIG. 35 is an enlarged view and cross-sectional view of a first embodiment of medule and nozzle assembly that uses the core portion of FIGS. 33-34 to be used with the mist spraying device of FIG. 28.

FIG. 36 is an enlarged view and cross-sectional view of the nozzle assembly of the medule and nozzle assembly of FIG. 35.

FIG. 37 is an enlarged view and cross-sectional view of a second embodiment of medule and nozzle assembly that uses the core portion of FIGS. 33-34 to be used with the mist spraying device of FIG. 28.

FIG. 38 is an enlarged view and cross-sectional view of the nozzle assembly of the medule and nozzle assembly of FIG. 37.

FIG. 39 shows a top portion of an embodiment of a nozzle assembly that can be used with the mist spraying device of FIG. 28.

FIG. 40 shows a perspective view of an embodiment of a container to be used with the nozzle assembly of FIG. 39.

FIG. 41a shows a side cross-sectional view of an alignment device to be used with the mist spraying devices of FIGS. 28-40.

FIG. 41b shows a front view of the alignment device of FIG. 41a.

FIG. 42 shows a perspective view of a new design of a mist spraying device in accordance with the present invention;

FIG. 43 shows an enlarged partial perspective view of the design of FIG. 42.

FIG. 44 shows a cross-sectional view of the mist spraying device of FIG. 42.

FIG. 45 is a left side view of the design of FIG. 42.

FIG. 46 is a right side view of the design of FIG. 42.

FIG. 47 is a front view of the design of FIG. 42.

FIG. 48 is a rear view of the design of FIG. 42.

FIG. 49 is a top view of the design of FIG. 42.

FIG. 50 is a bottom view of the design of FIG. 42.

FIG. 51 is a perspective view of the design of FIG. 42 with the label removed.

FIG. 52a is a front view of a container and cap to be used with the embodiments of FIGS. 28-34 and 39-48.

FIG. 52b is a cross-sectional view of the container and cap of FIG. 49b.

FIG. 53a is an exploded view of an embodiment of a medule top assembly to be assembly used with the embodiments of FIGS. 28-34 and 39-49b.

FIG. 53b is a perspective view of the medule top assembly of FIG. 50a.

FIG. 53c is a cross-sectional view of the bottle and cap of FIGS. 49a-b when attached with the medule top assembly of FIGS. 50a-b.

FIG. 54 is a cross-sectional view of a third embodiment of a medule and nozzle assembly when used with the mist spraying device of FIG. 28.

FIG. 55a is a perspective view of an embodiment of a closure system to be used with the misting device of FIGS. 42-51, wherein the diaphragm is in a closed position.

FIG. 55b is a perspective view of the closure system of FIG. 55a, wherein the diaphragm is in an open position.

FIGS. 56a)-c) show closed, partially open and fully opened positions, respectively, of the diaphragm of FIGS. 55a-b.

FIG. 57 is a cross-sectional view of an embodiment of a mesh plate protection device that can be used with the mist spraying devices of FIGS. 1-54.

### DETAILED DESCRIPTION OF THE PREFERRED EMBODIMENTS

Certain terminology is used in the following description for convenience only and is not limiting. As used herein, the term “distal” is meant to mean the discharge end of the inventive device and the term “proximal” is meant to mean the end of the inventive device held by user. The terminology includes the words above specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

The present invention provides a novel device and method for ophthalmic drug delivery. In preferred embodiments, the present invention provides a small, hand-held, battery or ac powered device that nebulizes liquid eye medications into a fine mist. The mist from the device is directed at the eye to be treated and the drug is delivered via the mist.

A preferred means of forming the mist is by ultrasound energy generated by a piezoelectric transducer or other suitable piezo device. A small plume of nebulized solution is generated, consisting of particles measuring what is believed to be in the range of five microns to twenty microns in diameter. The volume of each emission is dependent on the rate of mist generation (typically measured in micro liters per second) as well as the duration of the operation of the device, which may be easily varied by using an electronic control circuit. The shape, dimensions and focus of the emitted mist are proportioned for delivery to the human eye. The momentum of the mist is subliminal to the ocular blink and lacrimation reflexes and may also create a soothing sensation in the eye. The device is equally efficient when used in any “attitude” from a natural, upright head posture to leaning forward or lying back. Application time is significantly abbreviated compared to eye drop usage, which typically requires several maneuvers and careful attention to detail to ensure proper administration.

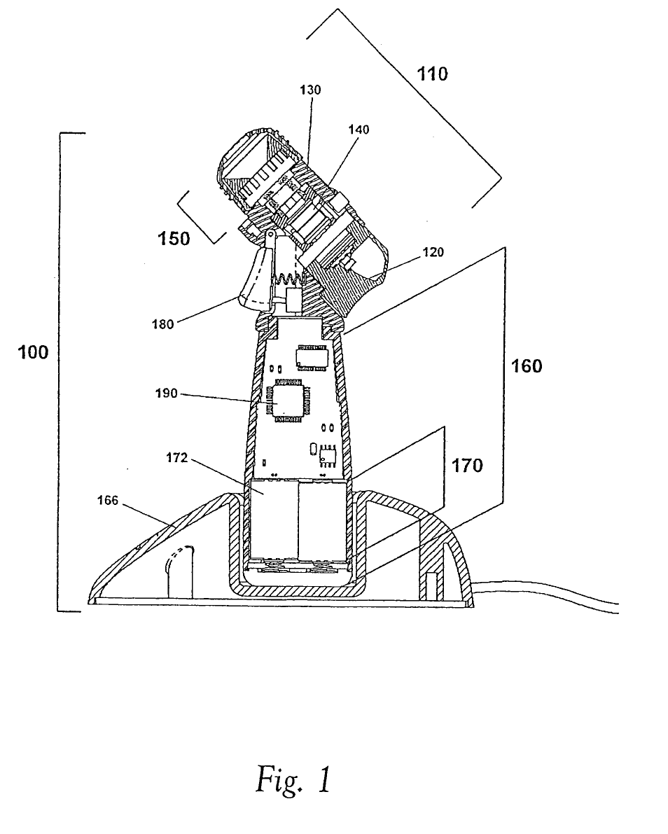

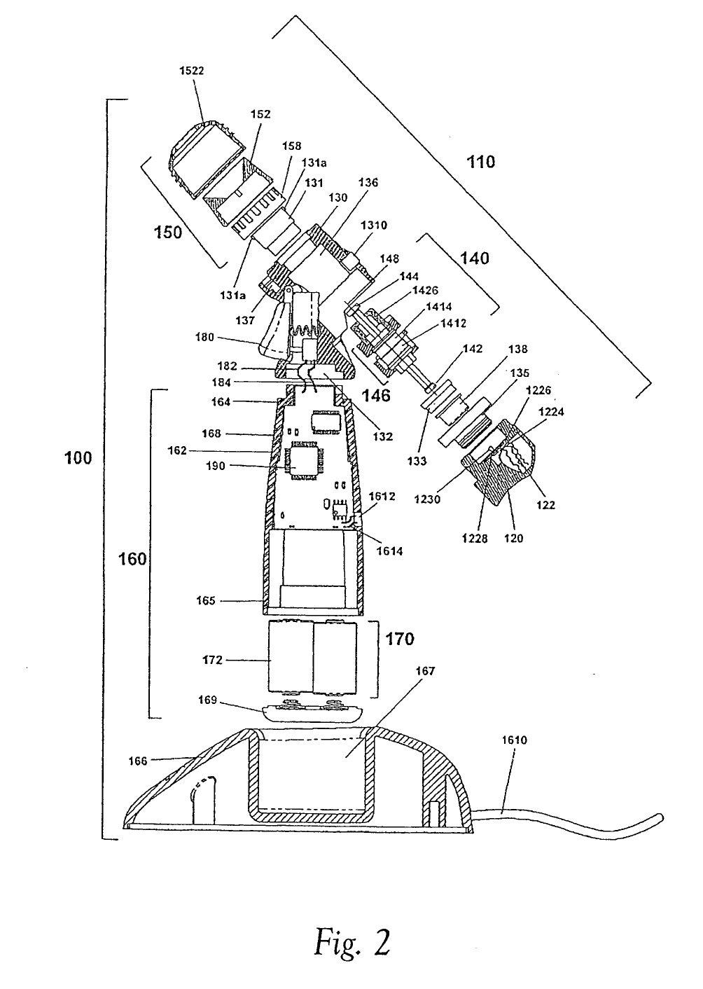

One preferred embodiment of the invention is now described with reference to FIGS. 1 and 2, which show a hand held device 100 that directs a mist of drug to an eye for treatment. As will be described in more detail below, the device 100 includes a vial or reservoir 120 of the fluid to be delivered to the eye, such as a drug. The user holds the device 100 and, by operating an activation switch, causes the device 100 to generate a mist of the liquid, which is discharged from the head portion 110 of the device 100. The user simply aims the head of the device at the target eye to allow the mist to contact the eye.

Referring to FIGS. 1 and 2, the major components of the device 100 are shown. The components include a head portion 110 and a handle portion 160. The head portion 110 preferably contains, from a proximal to a distal direction, a fluid reservoir 120 to retain a fluid 122 to be administered, a body 130 that houses a prime mover 140 to draw the fluid from the reservoir 120 and propel the fluid 122 out the distal end of the device 100, and a nozzle assembly 150 which aerosolizes the fluid 122 and to form a mist pattern of the fluid 122 as the fluid 122 is directed toward its target. The handle portion 160 preferably contains the power source 170, such as a battery, an activation switch 180 to activate the device, and a system controller 190 that controls the various operational aspects of the device 100.

### Head Portion

The head portion 110 includes the body 130 that connects the reservoir 120, the prime mover 140, and the nozzle assembly 150 together. The head portion 110 is connected to the handle portion 160 and provides a conduit for electrical leads (not shown) extending from the reservoir 120 and the prime mover 140 to the system controller 190.

### Reservoir

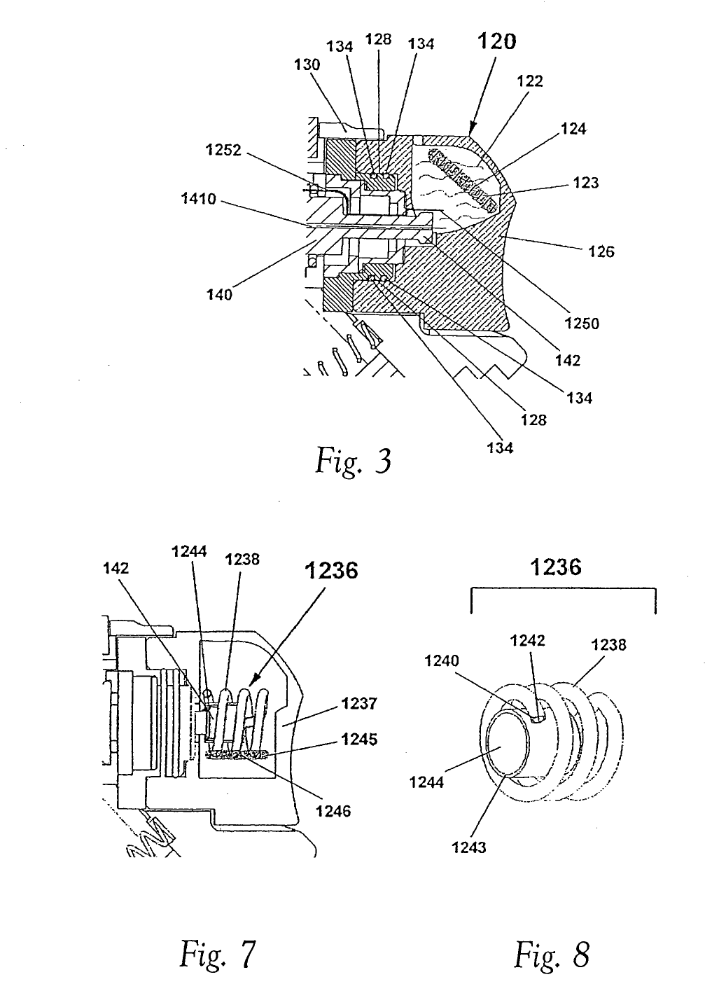

Referring to FIG. 3, in which an enlarged view of a preferred embodiment of the reservoir 120 is shown, the fluid reservoir 120 may can be a vial pre-filled with the fluid 122 to be delivered to the eye. The reservoir 120 may incorporate a scale comprising a clear window 123 with volume graduation markings 124 to indicate fill level or doses of fluid 122 remaining in the reservoir 120. In the present embodiment, the scale is read with the device 100 standing on its base 166, as shown in FIG. 1.

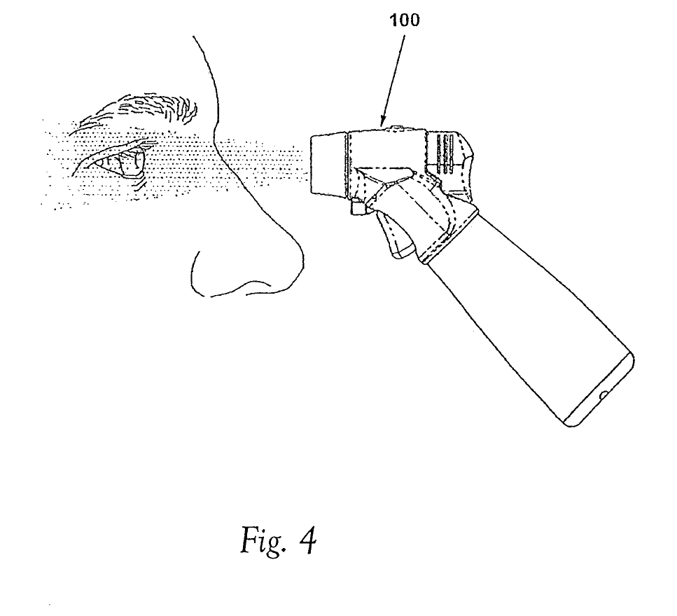

The reservoir 120 is preferably shaped to maintain contact with the prime mover 140 when the device 100 is held in a preferred operational orientation while spraying into an eye (as shown in FIG. 4), or is tilted in any direction within 45 degrees of horizontal. The reservoir 120 is preferably further shaped to maximize the percentage of the total fill volume that is actually dispensed.

Referring back to FIG. 3, the reservoir 120 houses the fluid 122 that is used to form the aerosolized mist when the device 100 is operated. The reservoir 120 is preferably a removable and replaceable cartridge 126 that is securably connectable to the body 130 so that the reservoir 120 does not accidentally readily separate from the body 120, yet is easily replaceable when the reservoir 120 is empty or when a reservoir 120 containing a different type of fluid is desired to be connected to the device 100.

Preferably, the reservoir 120 includes an engagement surface 128 disposed proximate to an upper and a lower side of the reservoir 120. The engagement surface 128 slides over a corresponding extension in the body 130, as shown in FIG. 3, so that the reservoir 120 is retained onto the body 130 with a frictional fit. Preferably, the extension includes a plurality of seals, such as o-rings 134, that provide a sealing engagement between the reservoir 120 and the body 130 and assists in frictionally retaining the body 120 to the reservoir 130. Alternatively, the reservoir 120 may connect with the body 130 by other means known to those skilled in the art, including, but not limited to, threaded connections, bayonet fittings, or other suitable means.

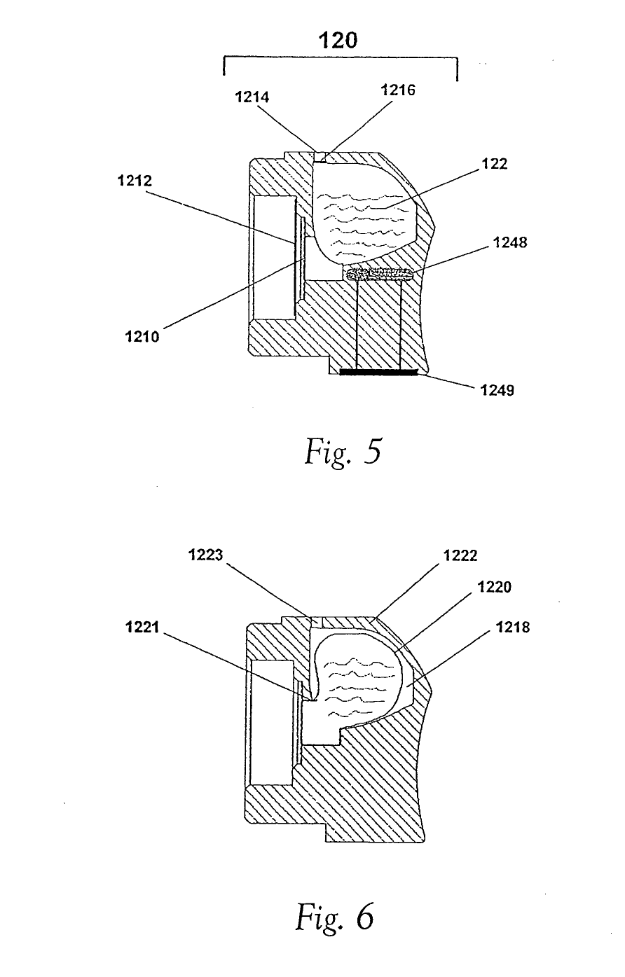

In the embodiment shown in FIG. 5, which shows the reservoir 120 removed from the remainder of the device 100, the reservoir 120 includes an open face 1210 that is covered by an air impermeable seal 1212. Initially, the open face 1210 allows the fluid 122 to be deposited into the reservoir 120, and then sealed with the seal 1212. Such a seal 1212 may be constructed from thin gauge aluminum, or some other suitable material, with a biocompatible coating disposed on both faces of the seal 1212. The seal 1212 is attached to the reservoir 120 with a biocompatible adhesive. The seal 1212 is designed to maintain sterility of the fluid 122 within the reservoir 120, yet be able to be easily punctured by the proximal end 142 of the prime mover 140 upon connecting the reservoir 120 to the body 130 so that the fluid 122 in the reservoir 120 is put into fluid communication with the proximal end 142 of the prime mover 140, as shown in FIG. 3.

For a reservoir 120 having a rigid form, such as the reservoir 120 shown in FIG. 5, a vent 1214 is formed in the wall of the reservoir 120, preferably proximate to the top of the reservoir 120, to allow air to be drawn into the reservoir 120 to compensate for the loss volume of fluid 122 as the fluid 122 is drawn out of the reservoir 120 due to operation of the device 100. A filter 1216 covers the vent 1214 to allow ambient air into the interior of the reservoir 120, but prevents fluid 122 in the reservoir 120 from leaking out of the vent 1214. While a presently preferred embodiment of the reservoir 120 envisions the fluid 122 to be prepackaged in the reservoir 120, those skilled in the art will recognize that the reservoir 120 may also be refillable, such as through the vent 1214.

Alternatively, as shown in FIG. 6, an alternate embodiment of a reservoir 1218 may have a collapsible bladder 1220 that collapses under vacuum as the fluid 122 is drawn out of the reservoir 1218 during operation of the device 100, without any air being able to enter the reservoir 122. The bladder 1220 is preferably supple, biocompatible, and bondable. In the presently preferred embodiment, the bladder 1220 is constructed of aluminum film coated on both sides with a polymer resin. In the presently preferred embodiment, the bladder 1220 is approximately 0.025 to 0.10 mm thick. The bladder 1220 is attached to a rigid bladder neck 1221. The neck 1221 prevents the bladder 1220 from contacting the proximal end 142 of the prime mover 140 as the bladder 120 collapses. Contact with the proximal end 142 would impede the function of the prime mover 140. The bladder neck 1221 may be injection molded or extruded from a material that is rigid, biocompatible, and bondable. A material which meets these criteria includes polyethylene, although those skilled in the art will recognize that other, suitable, biocompatible materials may be used. The bladder 1220 and bladder neck 1221 are housed in a rigid reservoir housing 1222. The housing 1222 is preferably injection molded from low cost polymer resins such as pvc, abs, or polypropylene.

An air vent 1223 in the housing 1222 allows the collapsible bladder 1220 to collapse as the fluid 122 is withdrawn from the reservoir 1218, so that no adverse suction forces are generated during operation of the device 100. The air entering the vent 1223 does not need to be filtered, since the bladder 1220 isolates the fluid 122 from the air. In this embodiment, no make-up air is required to enter the bladder 1220.

Without limiting the type of fluids that could be contained in the reservoir 120, 1218 and dispensed by the present invention, diagnostic agents used by the medical professional that could be delivered with the present invention include mydriatics/cycloplegics, anesthetics, flourescein and flouresceinlanesthetic combinations, and mydriatic reversal agents. Other agents which could be delivered with the present invention include over-the-counter agents, e.g., ophthalmic decongestants and lubricants, glaucoma medications (prostaglandins, beta blockers, alpha adrenergic agents, carbonic anhydrase inhibitors, miotics), and other ophthalmic medications. Optionally, several different therapeutic agents can be custom formulated in a single fluid to simplify adherence to multiple medication regimens.

Again, while an envisioned used for the device 100 of the present invention is directed toward ophthalmic use, those skilled in the art will recognize that the device 100 of the present invention may be used in other areas, such as respiratory treatment, and that other fluids, including respiratory medicaments, may be contained in the reservoir 120 instead.

Preferably, for photo-sensitive medicaments, the reservoir 120 may be tinted to prevent the transmission of certain deleterious wavelengths of light to the fluid 122 to prolong the useful life of the medicament in the reservoir 120. The tint may be a dark brownish tint that is presently used for such medicaments in bottle/eye dropper form.

Optionally, as shown in FIG. 2 the reservoir 120 may include a self-sealing valve 1224 in a distal wall 1226 of the reservoir 120. The self-sealing valve 1224 allows the reservoir 120 to be inserted into the body 130, and then removed from the body 130 without leaking fluid 122 from the reservoir 120.

The self-sealing valve 1224 is preferably biased toward a closed position, such as by a helical spring (not shown). A seal, such as an o-ring 1228, seals the valve 1224 against the wall 1226 of the reservoir 120 to eliminate fluid leakage from the reservoir 120 when the valve 1224 is in the closed position. A valve stem 1230 extends distally from the valve 1224. When the reservoir 120 is inserted into the body 130, the proximal end 142 of the prime mover 140 engages the valve stem 1230 and forces the valve stem 1230 into the reservoir 120, opening the reservoir 120 into fluid communication with the prime mover 140.

An alternative embodiment of a reservoir 1236 is shown in FIGS. 7 and 8. The reservoir 1236 is housed in a removable and replaceable cartridge 1237. The reservoir 1236 incorporates a generally coiled tube 1238 that is sized to partially surround the proximal end 142 of the prime mover 140. The tube 1238 may be constructed from polyethylene, although those skilled in the art will recognize that other suitable, biocompatible materials may be used. The tube 1238 preferably has a wall thickness in the range of approximately 0.1 to 0.3 mm thick, and an inside diameter in the range of approximately 1 to 5 mm. One end 1240 of the tube 1238 is fitted with a filter 1242 to allow makeup air to enter as the fluid 122 in the reservoir 1236 is drawn down. This filter 1242 is a biocompatible, gas-permeable membrane that is impermeable to liquid but permeable to air. One such material that may be used for the filter 1242 is Tyvek.®. A distal end 1243 of the tube 1238 is sealed with a fluid impermeable seal 1244 that is broken by the distal end 142 of the prime mover 140 when the reservoir 1236 is connected to the device 100, as shown in FIG. 7.

As the device 100 is operated and medication is consumed, the fluid 122 is drawn along the tube 1238. The diameter of the tube 1238 is preferably specified in relation to the viscosity of the fluid 122 to insure that surface tension causes the fluid 122 to move in a column along the tube 1238, i.e., no air is drawn in by the prime mover 140 until the fluid 122 is consumed. This design has the advantage of using nearly 100% of the medication loaded into the tube 1238. This configuration has the further advantage of allowing the device 100 to operate in any orientation, even in zero gravity environments. Referring to FIG. 7, a clear window 1245 and a numerical scale 1246 on the side of the cartridge 1237 may indicate how many doses remain in the reservoir 1236. The scale 1246 may be read with the device 100 in any orientation.

While a design of a reservoir 120 with a collapsible bladder 1220 and a design of a reservoir 1236 with a coiled tube 1238 are shown, those skilled in the art will recognize that other designs of reservoirs may be used.

Optionally, as shown in FIG. 5, a heater 1248 may be incorporated into the reservoir 120 to heat the fluid 122. The heater 1248 is preferably either an inductance or a resistive heater that is electrically connected to a contact 1249 in the wall of the reservoir 120 that is electrically connectable to a contact (not shown) in the body 130 to provide electrical power to the heater 1248 to heat the fluid 122 in the reservoir 120. However, for many ophthalmic medicines, heating the medicine is not desired, and those skilled in the art will recognize that the heater 1248 may be omitted in its entirety.

Also optionally, a low level sensor 1250, shown in FIG. 3, may be incorporated into the reservoir 120 to indicate when the fluid 122 in the reservoir 120 is almost depleted. The sensor 1250 is electronically connected to the system controller 190 via electrical connection 1252 to provide an indication of fluid level in the reservoir 120. The sensor 1250 may be electronically connected to an alarm, such as an optical or aural indicator, such as a blinking light or an audible alarm.

### Body

Referring back to FIG. 2, the body 130 houses the prime mover 140 and provides a connection for the fluid reservoir 120 and for the nozzle assembly 150 to engage the prime mover 140. The body 130 includes, at the distal end of the body 130, a bushing 131 that is securely bonded to the body 130, such as by an adhesive or a snap-fit. The bushing 131 includes at least one, an preferably, a plurality of bayonet clips 131a that are adapted to snap into the nozzle assembly 150 to retain the nozzle assembly 150 onto the body 130.

The body 130 preferably includes a connection device, such as an orifice 132, for attaching to the handle portion 160. However, those skilled in the art will recognize that other connection methods, such as snap fit, bayonet clips, or other suitable mechanisms known to those skilled in the art may be used. Preferably, the body 130 connects to the top 162 of the handle portion 160 in only a single orientation so that electrical contacts in each of the body 130 and the handle portion 160 properly engage each other when the head portion 110 is connected to the handle portion 160.

The body 130 also includes, at the proximal end of the body 130, a collar spacer 133 that is fixedly connected to the body 130 to provide optimum spacing of the proximal end 142 of the prime mover 140 within the reservoir 120 to optimize the ability of the prime mover 140 to withdraw the fluid 122 from the reservoir 120 during operation of the device 100.

The body 130 houses the prime mover 140, and provides connection means for the reservoir 120, the nozzle assembly 150, and the handle portion 160. The retainer 135 is fixedly connected to the body 130 and also releasably retains the reservoir 120 so that the reservoir 120 is removable from the remainder of the device 100. As described above, the retainer 135 may include an engagement surface, or alternatively, other connection means, such as threaded connections, or other means known to those skilled in the art.

The body 130 includes a generally tubular passage 136 that is sized to accept the proximal end 142 of the prime mover 140. A spacer recess 137 is disposed at the distal end of the body 130, preferably below the passage 136. The spacer recess 137 is used to releasably retain a targeting means, which will be described in detail later herein.

A seal 138 is disposed about the proximal end of the passage 136. The seal 138 prevents any fluid 122 from leaking out of the reservoir 120 when the reservoir 120 is attached to the body 130. In the present embodiment, the seal 138 is formed in the shape of a ring by injection molding or liquid injection molding using medical grade silicones or urethanes with durometers in the range of 5 to 30 Shore A.

Preferably, the body 130 includes an activation indicator 1310 that is disposed on the top of the body 130. The activation indicator 1310 may be a light, such as an led, that provides constant illumination as long as the activation switch 180 is depressed; a light that provides blinking illumination; a sound that provides audible indication, either by constant or by periodic beeping; some combination of these listed indicators, or some other indication that would indicate to the user that the device is ready for operation. The activation indicator 1310 operates when the activation switch 180 is initially depressed by the user. The activation indicator 1310 alerts the user that the device 100 is “on” and is about to spray the fluid 122 from the nozzle assembly 150. The activation indicator 1310 is electronically connected to the system controller 190 via electrical leads (not shown).

The body 130 may be machined from solid metal or plastic stock, or may be injection molded with polymer resins such as abs, styrene, pvc, or other suitable material, as will be recognized by those skilled in the art. The body 130 may be injection molded or manufactured by other methods known by those skilled in the art. Preferably, the body 130 has a durometer within the range of approximately 90 to 100 Shore A.

### Prime Mover

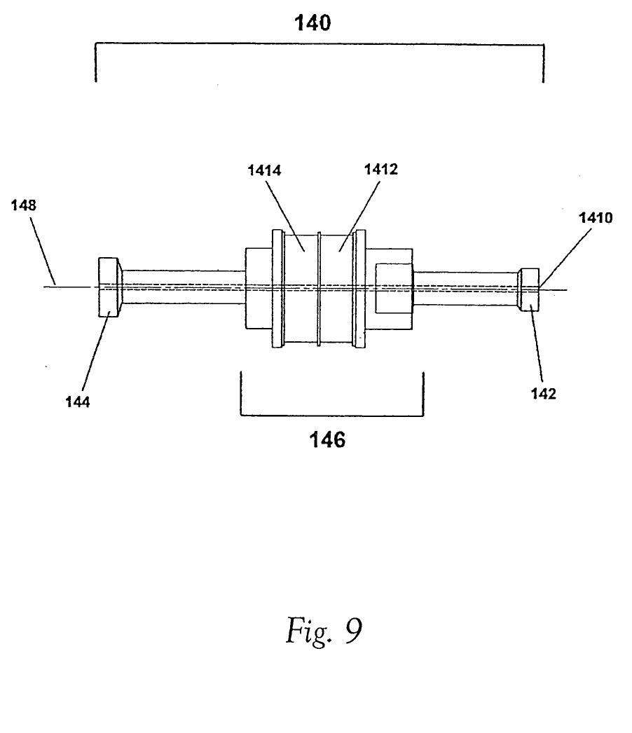

Referring still to FIG. 2, as well as to FIG. 9, the prime mover 140 will now be described. The prime mover 140 is shown in FIG. 2 in relation to the nozzle assembly 150 and the reservoir 120. The prime mover 140 is preferably an ultrasonic oscillator formed by a piezoelectric assembly such as that found in the Omron Micro-air Model NE-U03. The NE-U03 is a commercially available nebulizer that is typically used in nebulizers for bronchial therapy. However, the inventors of the present invention have discovered that this particular nebulizer is also suited for delivery of ophthalmic medicine to satisfy the needs that the present invention is intended to satisfy. The preferred piezoelectric assembly is described in detail in U.S. Pat. No. 6,651,650, the disclosure of which is incorporated herein by reference. However, those skilled in the art will recognize that the NE-U03 may be substituted for other piezoelectric assemblies, such as those discussed in the article “Nebulizers that use a vibrating mesh or plate with multiple apertures to generate aerosol,” by Rajiv Dhand MD, Respiratory Care, December 2002, vol. 47, no. 12, which is also incorporated by reference herein. Alternatively, instead of using piezoelectric assemblies, those skilled in the art will recognize that other prime movers that are not piezoelectrically operated may be used. Examples of such other suitable prime movers include electric pumps, manual pumps, compressed gas, or other suitable prime movers, as will be recognized by those skilled in the art.

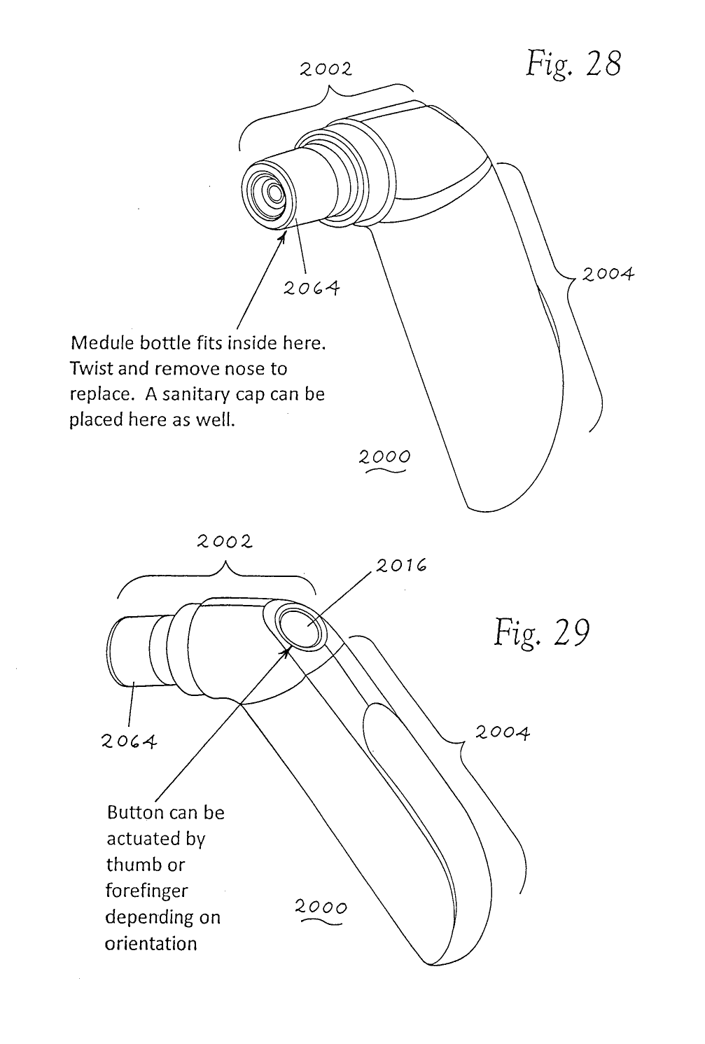

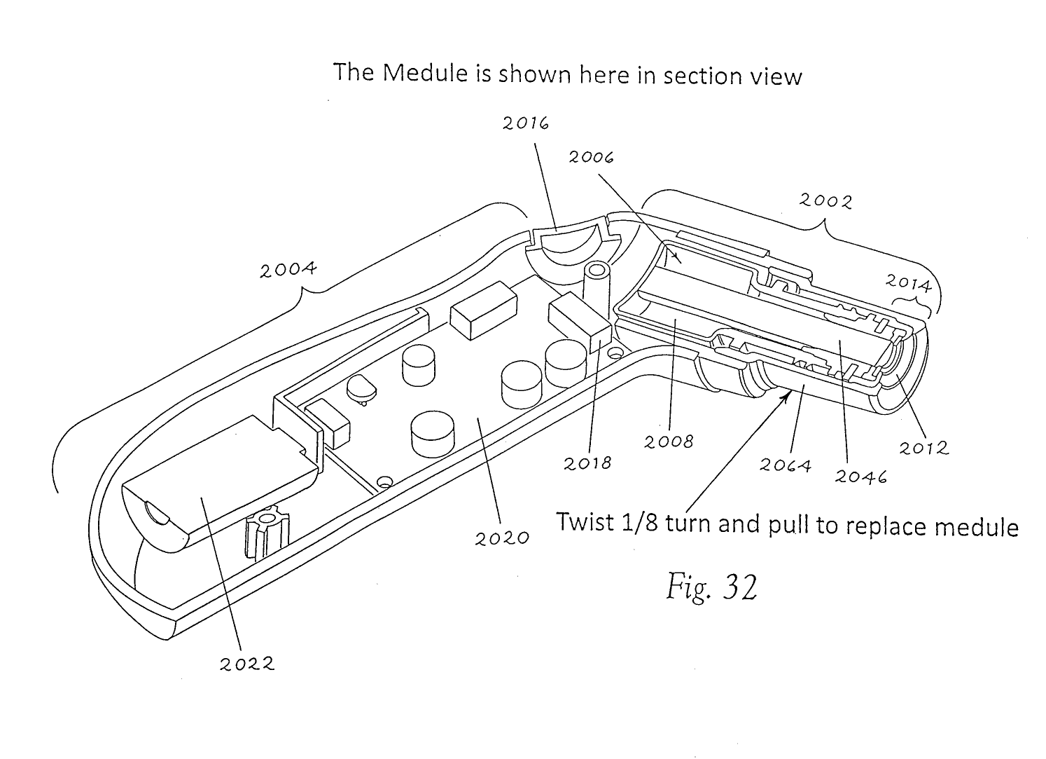

The prime mover 140 includes a proximal end 142, a distal end 144, and a central portion 146 disposed between the proximal end 142 and the distal end 144. A longitudinal axis 148 extends along a length of the prime mover 140 between the proximal end 142 and the distal end 144. A longitudinally extending lumen 1410 extends along the longitudinal axis 148 and extends the length of the prime mover 140. Preferably, a perpendicular cross section of the lumen 1410 is generally circular in shape and has a diameter of approximately between 0.25 and 1.0 mm. However, those skilled in the art will recognize that the lumen 1410 may have other cross sectional shapes, such as a generally oblong, oval, or elongated shape. As an alternative embodiment, the lumen 1410 can be replaced by a wick that has the same dimensions as the wick 1410. More details regarding the wick are discussed with respect to the embodiments of FIGS. 28-38.

The central portion 146 includes at least two generally annular piezoelectric elements 1412, 1414 that surround the lumen 1410. The piezoelectric elements 1412, 1414 are electrically connected to the power source 170, which drives the piezoelectric elements 1412, 1414 during operation of the device 100.

Referring to FIG. 2, the prime mover 140 is retained within the body 130 by a distal seal 1426. The distal seal 1426 is generally annular in shape and taper from a wider diameter to a smaller diameter from the piezoelectric elements 1412, 1414 toward the proximal end 142 and the distal end 144, respectively. The distal seal 1426, along with the seal 138, restricts movement of the prime mover 140 within the body 130 and prevent fluid 122 that may leak through the device 100 from engaging the central portion 146 of the prime mover 140. Preferably, the seal 1426 is constructed from a biocompatible material, such as medical grade silicon or urethane, although those skilled in the art will recognize that other suitable material may be used.

Referring back to FIG. 3, the proximal end 142 is immersed in the fluid 122 in the reservoir 120. In one theory of operation, when the piezoelectric elements 1412, 1414 are excited, such as during operation of the device 100, standing waves are formed which draw the fluid 122 into the proximal end 142 of the prime mover 140 and along the lumen 1410. The standing waves propel the fluid 122 along the lumen 1410 to the distal end 144 of the prime mover 140 and to the nozzle assembly 150, which is in mechanical contact with the distal end 144 of the prime mover 140. As the prime mover 140 vibrates at ultrasonic frequencies, the prime mover 140 transfers a portion of its vibrational power to a mesh plate 156 in the nozzle assembly 150, as will be described in more detail later herein. In another theory of operation, the size of the lumen is such that fluid is drawn up the lumen by capillary action. In either theory of operation, the fluid 122 that has been propelled along the lumen 1410 contacts the mesh plate 156. The vibration of the plate 156 aerosolizes the fluid 122 and accelerates the fluid 122 away from the device 100 and toward the patient.

Note that in order to produce vibrations in the plate 156 of sufficient amplitude in an efficient manner, the prime mover geometry is chosen so as to amplify oscillations produced in opposing piezoelectric elements 1412, 1414. Thus, when the prime mover 140 is driven at its natural frequency (resonance), the amplitude generated at the plate 156 is greater when compared to the energy input.

### Nozzel Assembly

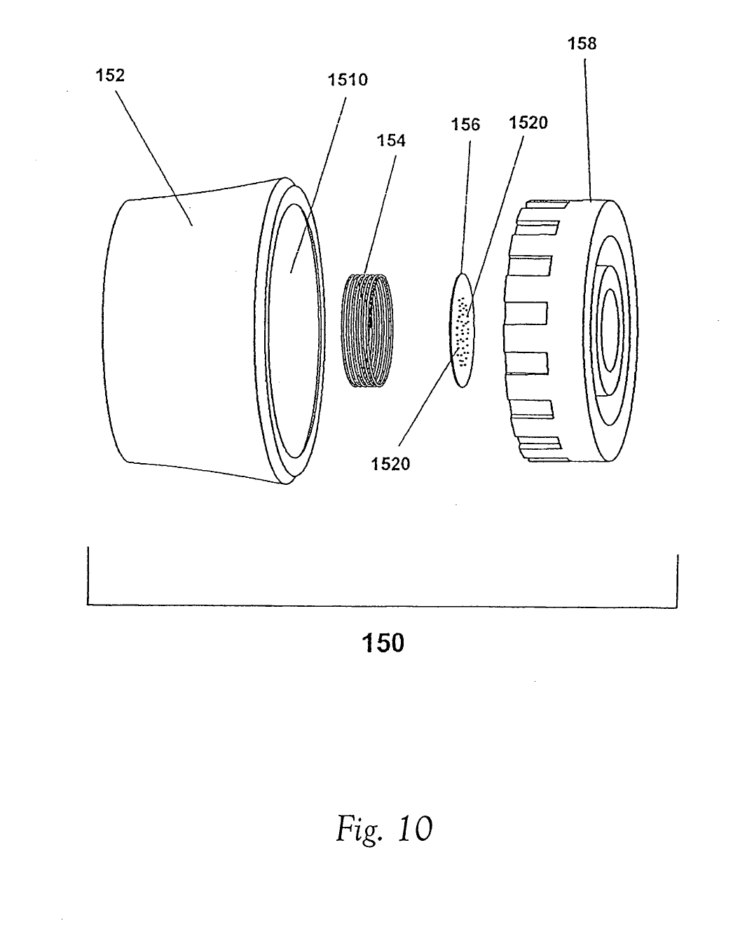

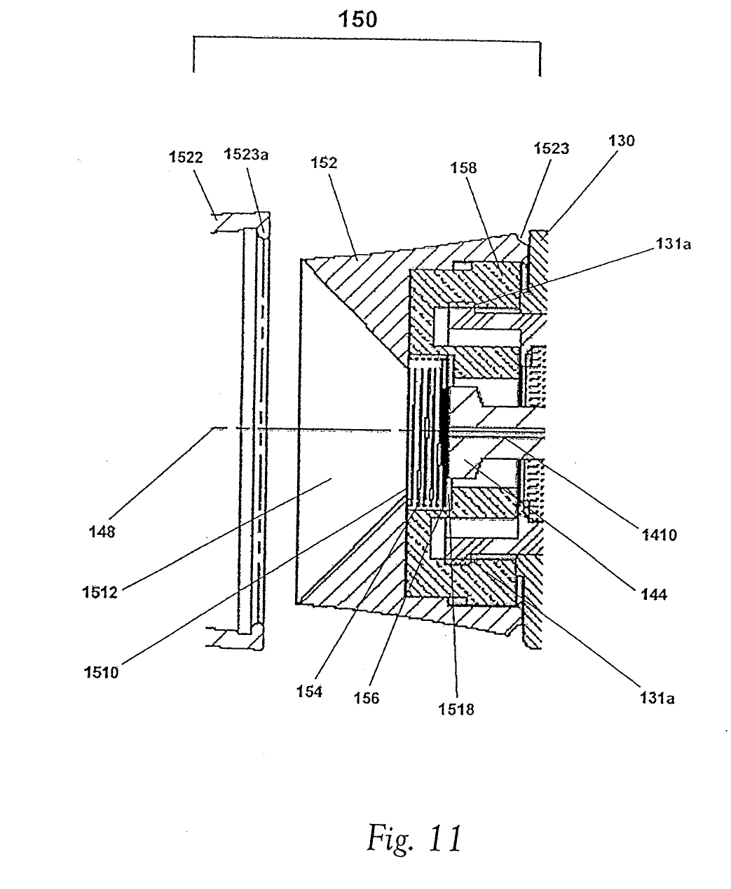

The nozzle assembly 150 is shown in an exploded perspective view in FIG. 10, as well as in an elevated sectional view in FIG. 11. The nozzle assembly 150 forms the mist that is discharged from the device 100 during operation. The nozzle assembly 150 includes, from a distal to a proximal direction, a cap 152, a biasing member 154, a mesh plate 156, and a retainer 158.

The cap 152 is generally annular, with a central opening 1510 disposed along the longitudinal axis 148. Preferably, the body of the cap 152 extends in a distal direction and generally away from the longitudinal axis 148 to form a concave volume 1512 distal of the central opening 1510. The concave volume 1512 reduces the likelihood that a foreign object, such as a user's finger, will touch the mesh plate 156, potentially contaminating the plate 156.

The cap 152 preferably includes a releasable lock feature, such as a female threaded connection (not shown) that releasably threadingly engages the retainer 158, which has a mating twist lock feature, such as a mating male threaded connection (not shown). However, those skilled in the art will recognize that the cap 152 may engage with the retainer 158 by other means not shown, such as by snap engagement, bayonet means, or other suitable means known to those skilled in the art.

The mesh plate 156 is biased against the distal end 144 of the prime mover 140 by the biasing element 154, such as a helical spring, that is disposed between the cap 152 and the mesh plate 156. The biasing element 154 ensures that the mesh plate 156 is firmly engaged with the distal end 144 of the prime mover 140 to provide proper dispersion of the fluid 122 through the mesh plate 156 during operation of the device 100. While a helical spring is preferred as the biasing element 154 because a helical spring provides a generally uniform biasing force around its perimeter, those skilled in the art will recognize that other types of biasing elements, such as leaf springs, may be used instead. As shown in FIG. 11, a clearance space 1518 is formed between the proximal side of the mesh plate 156 and the retainer 158 to allow the mesh plate 156 to vibrate during operation.

The mesh plate 156 is formed of a rigid material that is biocompatible and non-oxidizing, such as alumina ceramics, Palladium Nickel, titanium alloys, or stainless steel alloys. As shown in FIG. 10, an array of openings 1520 is formed in the mesh plate 156. The number, density, size, and shape of the openings 1520 contribute to determining mist parameters such as volume, velocity, and droplet size distribution. The openings 1520 may be drilled by mechanical means, by fine jets of water, or by lasers. In the alternative, the openings 1520 can be formed by electroforming, wherein the mesh plate 156 and the openings 1520 are formed simultaneously with a plating-type operation. The preferred embodiment of the mesh plate 156 is constructed from a ceramic material and measures approximately 9 mm in diameter and 0.1 mm thick, having between 500 and 5000 openings 1520 drilled by laser. The openings 1520 preferably have a shape of a cymbal and have diameters in the range of approximately 0.5 to 30 microns. A mask (not shown) may be used that enables many openings 1520 to be drilled simultaneously. After each group of openings 1520 is drilled, the mask or the mesh plate 156 is indexed to a new position and the next set of openings 1520 is drilled. This step-and-repeat process continues until all the openings 1520 are made.

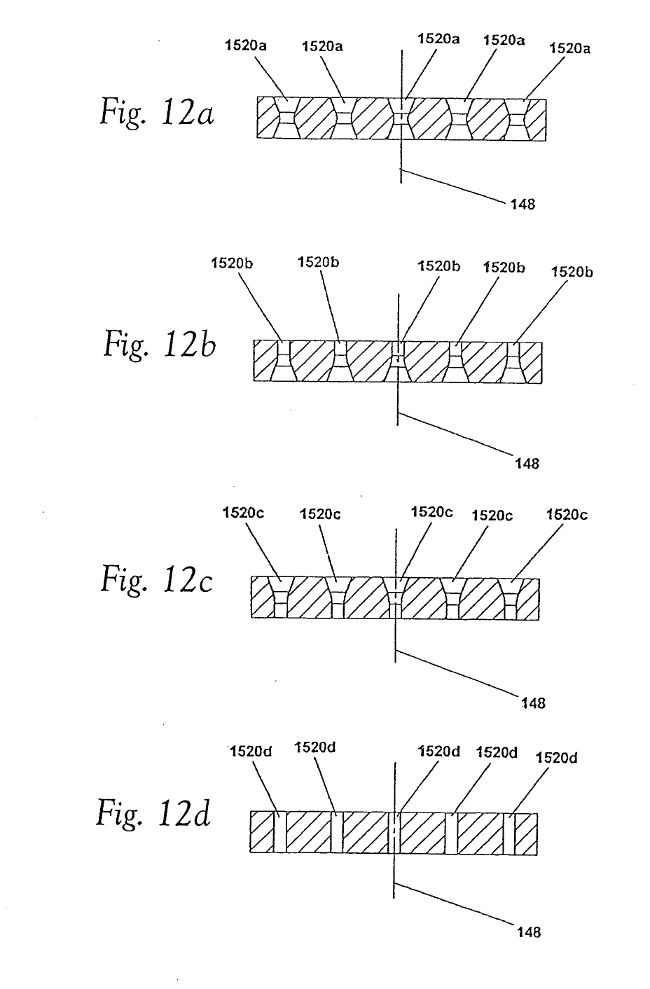

Enlarged cross sections of several embodiments of openings 1520a, 1520b, 1520c, 1520d, and 1520e in mesh plates 156a, 156b, 156c, 156d, 156e are shown in FIGS. 12a, 12b, 12c, 12d, 12e. Referring to FIG. 12a, the mesh openings 1520a in the mesh plate 156a are preferably circular in cross section along a plane parallel to the longitudinal axis 148, with an approximate hourglass cross section along a plane perpendicular to the longitudinal axis 148. Referring to FIG. 12b, the mesh openings 1520b in the mesh plate 156b are wider at the proximal (bottom) end of the plate 156b and narrower at the distal (top) end of the plate 156b. Referring to FIG. 12c, the mesh openings 1520c in the mesh plate 156c are narrower at the proximal (bottom) end of the plate 156c and wider at the distal (top) end of the plate 156c. Referring to FIG. 12d, the mesh openings 1520d in the mesh plate 156d have a generally constant diameter between the proximal (bottom) end of the plate 156d and the distal (top) end of the plate 156d.

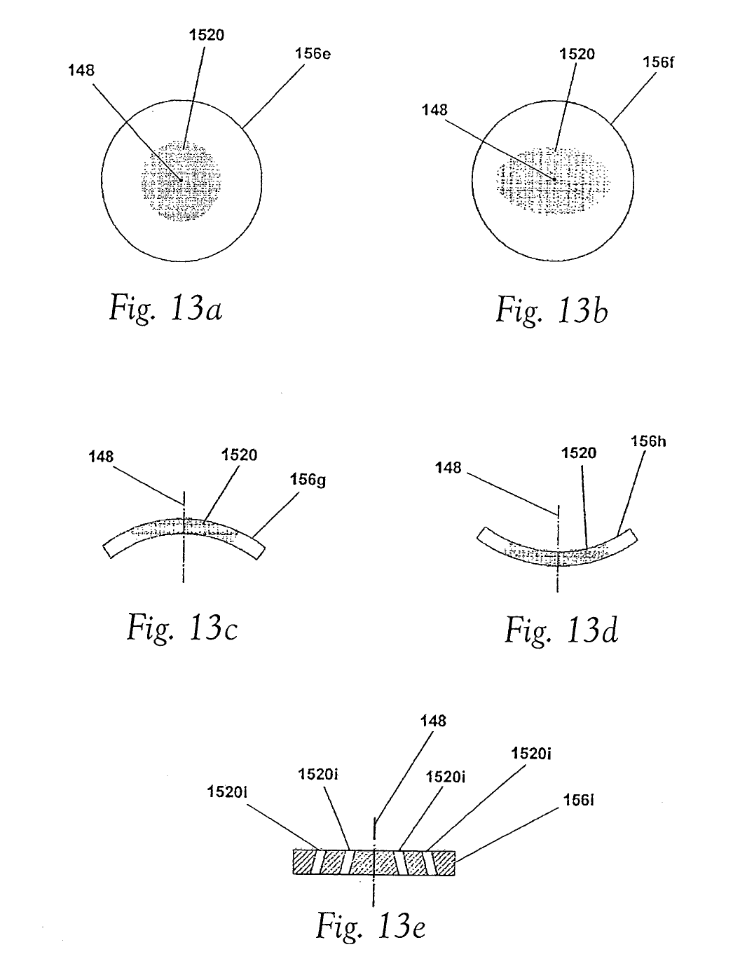

The mesh plate may 156 incorporate one of several designs of openings 1520 as shown in FIGS. 13a through 13e. In the top plan view of the design shown in FIG. 13a, a mesh plate 156e is generally planar, with a plurality of openings 1520 in a generally circular pattern, with a center of the generally circular pattern along the longitudinal axis 148. In the top plan view of the design shown in FIG. 12b, a mesh plate 156f is generally planar, with a plurality of openings 1520 in a generally elongated pattern, such as a rectangle or an oval. Alternatively, a mesh plate 156g may be generally convex, as shown in the side sectional view of the mesh plate 156g in FIG. 13c, to disperse the fluid 122 at a relatively wide angle to increase the field of dispersion of the fluid 122. In yet another alternative, a mesh plate 156h may be concave, as shown in the side sectional view in FIG. 13d, to disperse the fluid 122 in a relatively small area. For each of the mesh plates 156g, 156h in FIGS. 13c and 13d, the pattern of openings may be circular, as shown in FIG. 13a, or elongated, as shown in FIG. 13b. The pattern of openings 1520 is aligned with the central opening 1510 in the cap 152 so that the fluid 122 that is dispersed through the mesh plate 156 passes through the central opening 1510 and forms a mist for deposition into the eye of the patient.

In an alternate embodiment, shown in Fig. Be, a mesh plate 156i includes a generally flat plate with openings 1520i that are angled toward the longitudinal axis 148. This design provides the benefits of an easy to produce mesh plate that directs the fluid to a focused point.

It is preferred that the openings 1520 in the mesh plate 156 generate mist particle sizes in the average range of between approximately 0.5 and 20 microns in diameter. It is also desired that the mist generated through the nozzle assembly 150 preferably extends about 7.5 to 10 cm in a mist plume diverging with a solid angle of approximately 10-20 degrees and traveling at a velocity of between approximately 4 and 30 cm per second, discharging approximately between 2 and 20 microliters per second, and preferably, between 7 and 10 microliters of fluid per second.

Referring back to FIG. 11, the retainer 158 preferably connects to the body 130 via the plurality of bayonet fittings 131a that snap into the retainer 158, although those skilled in the art will recognize that other means for connecting the retainer 158 to the body 130, such as by threaded connection, adhesive, or other suitable means, may be used.

The mesh plate 156 is removable from the remainder of the device 100 for cleaning, such as in an alcohol or other cleaning solution. To clean the mesh plate 156, the retainer 158 is removed from the body 130, releasing the cap 152, the biasing element 154, the mesh plate 156, and the retainer 158 from the remainder of the device 100. The biasing element 154 biases the mesh plate 154 against the retainer 158, keeping the nozzle assembly 150 intact. After cleaning, the nozzle assembly 150 is reconnected to the remainder of the device 150. The distal end 144 of the prime mover 140 engages the mesh plate 156, forcing the mesh plate 156 away from the retainer 158 so that the mesh plate 156 may be able to vibrate when excited by the prime mover 140.

Optionally, as shown in FIGS. 2 and 11, an overcap 1522 may be disposed over the distal end of the cap 152 to keep the mesh plate 156 clean between uses. The cap 152 may include a peripherally spaced groove 1523 that is engageable with a corresponding protuberance 1523a for a snap fit connection that securely retains the overcap 1522 onto the cap 152, yet allows the overcap 1522 to be removed from the cap 152 with a minimum of effort. Alternatively, the overcap 1522 may attach to the cap 152 with a snap action, a thread, a bayonet, or other simple fastening means. The overcap 1522 may be machined from solid metal or plastic stock, or may be injection molded with polymer resins such as abs, styrene, or pvc. The overcap 1522 may optionally be tethered to the device 100 with a lanyard made of wire cable or plastic filament. Alternatively, the overcap 1522 may be attached to the nozzle assembly 150 with a hinge (not shown). The hinge may incorporate a spring or other biasing member that automatically retracts the overcap 1522 away from the distal end of the cap 152 when a latch is released.

### Dosage Adjustment

Different medications and/or ophthalmic treatment regimens may require different amounts of a medication to be administered with each use of the device 100. Alternatively, a larger patient may need a larger dose of a medication than a smaller patient. Therefore, an ability to adjust dosage amount may be required. The device 100 may optionally be equipped with user-accessible adjustments for flow rate (mist volume) and total flow (dose). These adjustments may be electro-mechanical (knobs or wheels operating potentiometers), or electronic (buttons or keys providing digital data to the system controller 190).

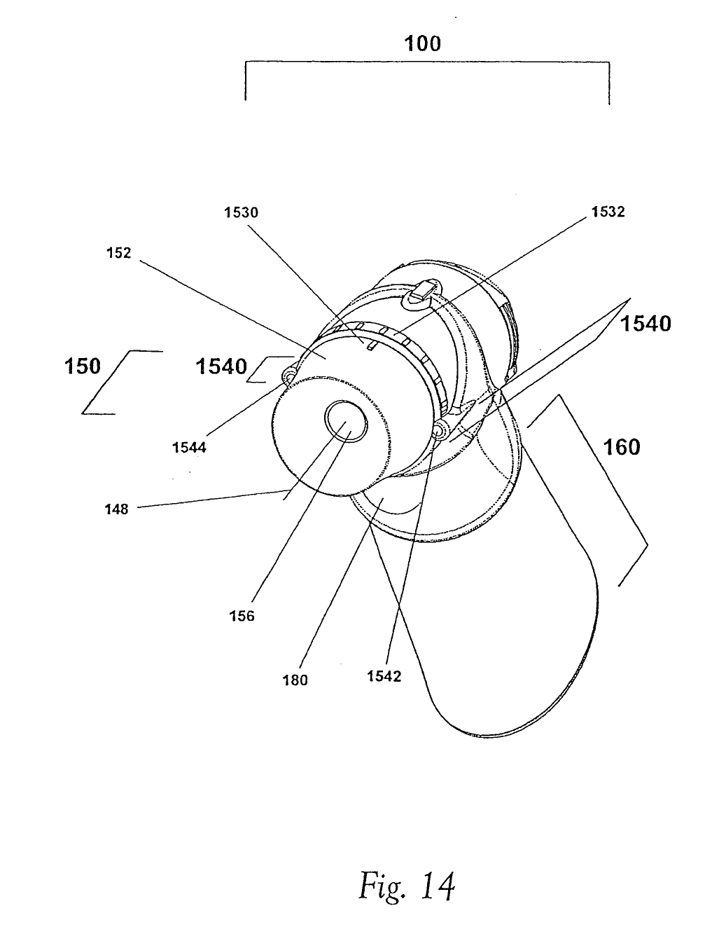

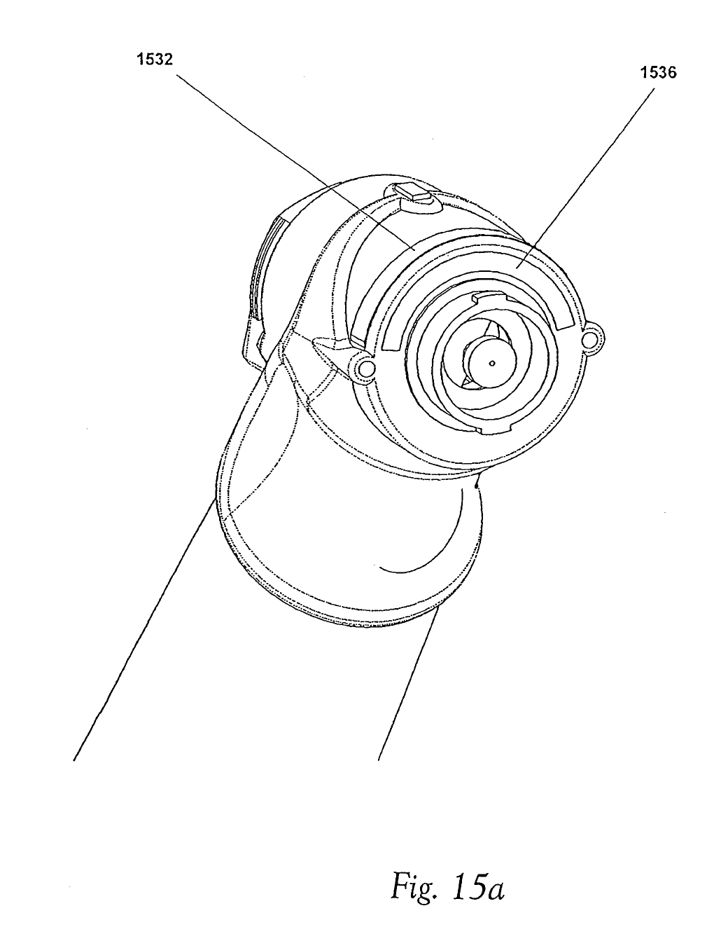

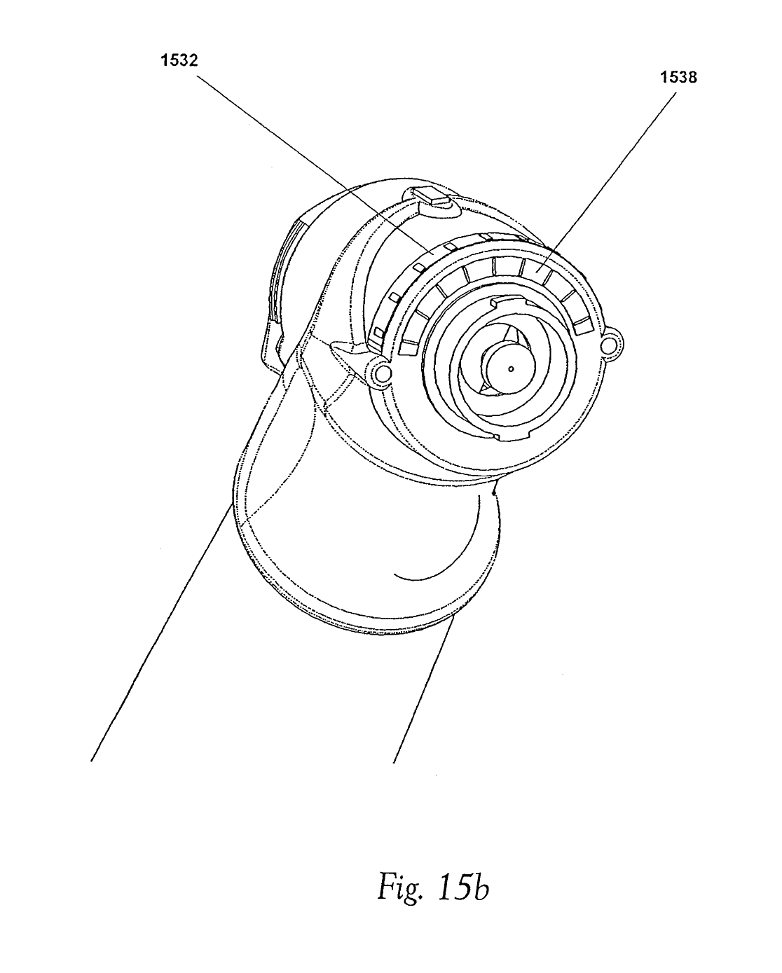

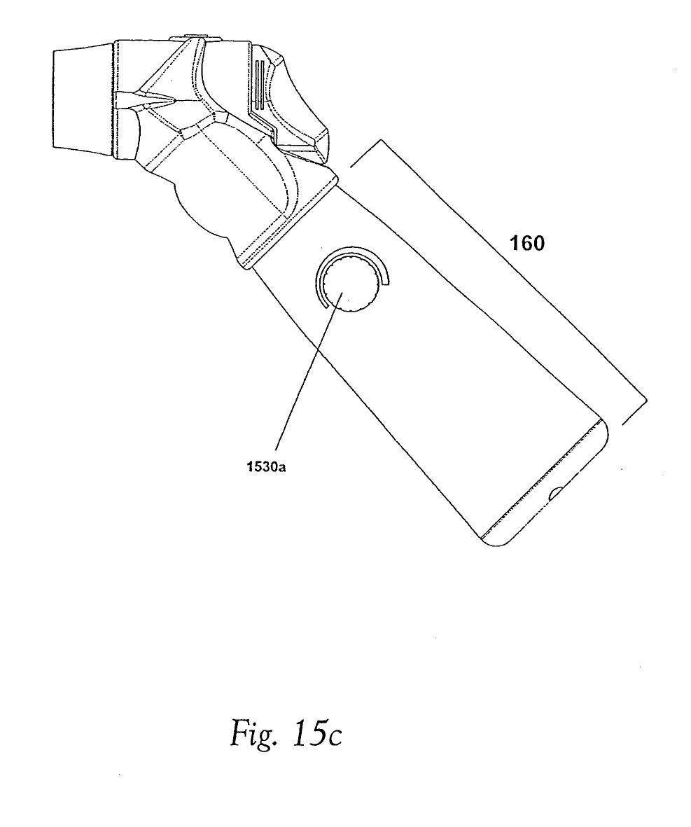

In one embodiment of a dosage adjustment, a dosage adjuster 1530, 1530a may be disposed on the nozzle assembly 150, such as is shown in FIGS. 14 and 15a-15b. The dosage adjuster 1530 includes a potentiometer 1532 rotatably connected to the cap 152. The potentiometer 1532 may include an infinitely positionable pot that is movable across a resistive film 1536, as shown in FIG. 15a, or a discretely positionable pot that is movable across a resistive film 1538 as shown in FIG. 15b. For either film 1536, 1538, rotation of the potentiometer 1532 changes the resistance of the potentiometer circuit, as is well known to those skilled in the art. The change in resistance changes a dosage voltage signal that is transmitted to the system controller 190 via a circuit (not shown). The system controller 190 interprets the voltage signal received and in turn transmits an operation duration signal to the prime mover 140, which controls the amount of time that the prime mover 140 operates when the activation switch 180 is engaged, thereby controlling the amount of fluid 122 that is discharged from the device 100.

While the dosage adjuster 1530 may be disposed on the nozzle assembly 150 as shown, those skilled in the art will recognize that a dosage adjuster 1530a may be disposed on the handle portion 160, as is alternately shown in FIG. 15c. The dosage adjuster 1530a preferably operates similarly to the dosage adjuster 1530 described above. Preferably, the dosage adjuster 1530a is disposed in an inconvenient location, such as behind a panel (not shown). It is typically not desirable to be able to easily adjust the dosage adjuster 1530a so that the user does not accidentally adjust the dosage while picking up or holding the device 100. The volume of fluid 122 dispensed as a mist from the device 100 is preferably adjustable between about 10 to 100 microliters by adjusting the duration of time at which the mist is supplied.

It is envisioned that other ways can be employed to adjust the flow rate. For example, the peak voltage supplied to the prime mover 130 can be adjusted so as to control the amplitude of mesh plate oscillation, thereby controlling flow rate. Another possibility is to employ pulse width modulation to control the flow rate. In such a process, periodic pulses are supplied to the prime mover 130, wherein the prime mover 130 is energized by the amount of time equal to the pulse width. By adjusting the value of the pulse width, the amount of time the prime mover is energized during a pulse can be varied. Note that the pulse width modulation process can be varied to generate pulse trains that generate small packets of mist that are sequentially delivered to the eye. For example, the pulse timing could be 0.2 seconds on, 0.3 seconds off, repeated 4 times. The 4 packets of mist generated appear to disrupt the formation of eddies that can cause some particles to miss the eye and/or face entirely. Of course, other combinations of mist packets are possible.

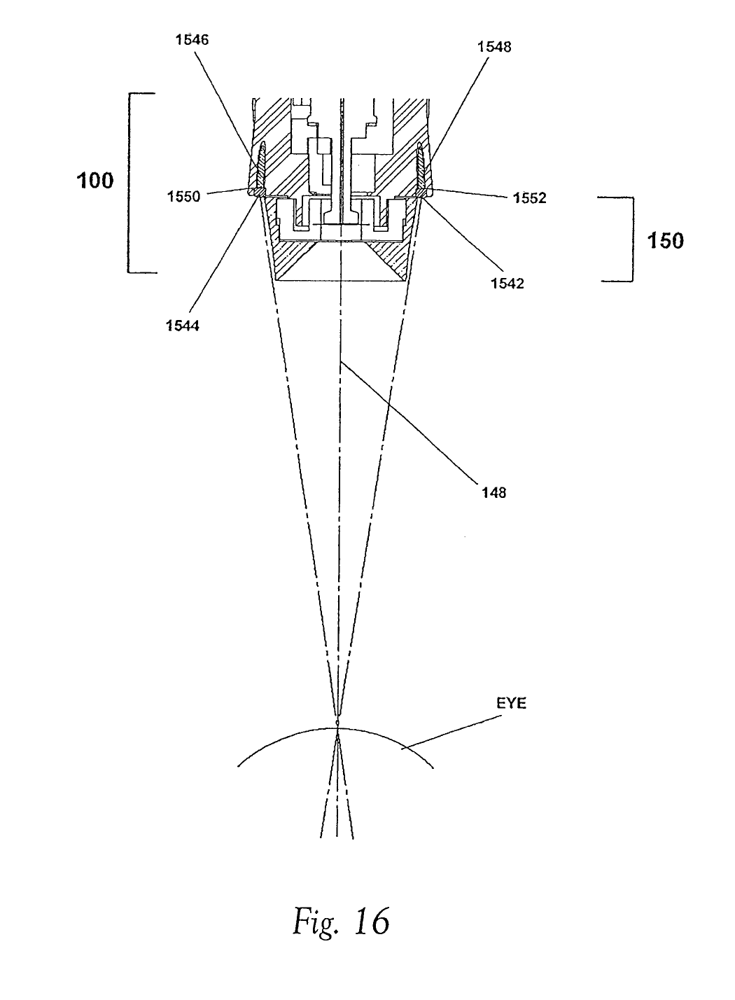

In order to ensure that dosing is consistent, the location of the nozzle assembly 150 relative to the eye during dispensing of medication may also need to be controlled. Various targeting mechanisms have been developed for this purpose. Referring back to FIG. 14, a first embodiment of a targeting mechanism 1540 may be incorporated into the nozzle assembly 150. The targeting mechanism 1540 is used to provide the user with an optimum distance to space the nozzle assembly 150 from the patient's eye to maximize effectiveness of the device 100. The targeting mechanism 1540 includes two projection lenses 1542, 1544 that are disposed on the nozzle assembly 150, preferably spaced 180 degrees from each other on either side of the longitudinal axis 148. The lenses 1542, 1544 are angled toward the longitudinal axis 148 such that projections from the lenses 1542, 1544 intersect at the longitudinal axis 148 at an optimum distance for spacing the nozzle assembly 150 from the patient's eye, as shown in FIG. 16. A light source 1546, 1548 is disposed proximal of each lens 1542, 1544, respectively, with each light source 1546, 1548 being directed along the projection line of each respective lens 1542, 1544. The light sources 1546, 1548 may be LEDs, incandescent sources, lasers, or other suitable light source, as will be recognized by those skilled in the art. The light sources 1546, 1548 are electrically connected to the activation switch 180 so that the light sources 1546, 1548 activate upon initial engagement of the activation switch 180.

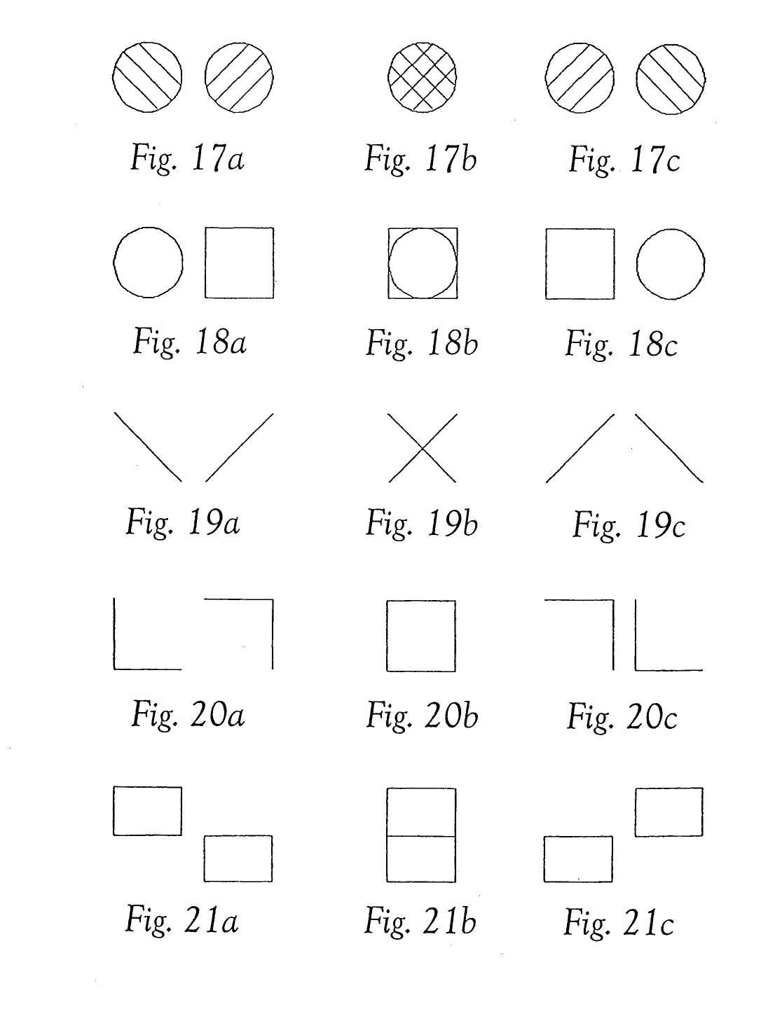

Preferably, the light sources 1546, 1548 and the lenses 1542, 1544 form a pattern on the target eye when the device 100 is aimed at the eye and the activation switch 180 is depressed. The pattern may be formed by separate masks 1550, 1552 that are disposed between each light source 1546, 1548 and its respective lens 1542, 1544, as shown in FIG. 16, or, alternatively, the mask may be formed on each lens 1542,544 (not shown). In either embodiment, the targeting mechanism 1540 forms one of three general patterns on the iris or the sclera of the eye. When the device 100 is too far from the eye, a pattern similar to a pattern formed in one of FIGS. 17a, 18a, 19a, 20a, 21a is formed. When the device 100 is a correct distance from the eye, a pattern similar to the pattern formed in one of FIGS. 17b, 18b, 19b, 20b, 21b is formed. When the device 100 is too close to the eye, a pattern similar to the pattern formed in one of FIGS. 17c, 18c, 19c, 20c, 21c is formed. Those skilled in the art will recognize that the patterns shown in FIGS. 17a-21c are exemplary only, and that numerous other patterns may be formed.

In addition to assisting in determining the optimum distance for spacing the device 100 from the eye, the targeting mechanism 1540 also aids in accurately aiming the device 100 at the eye, so that the mist generated by the device 100 is directed toward the middle of the eye, and not off to the side.

While the targeting mechanism 1540 described above is useful for a professional practitioner to use to aim the device 100 at a patient, those skilled in the art will recognize that an alternative embodiment of a targeting mechanism (not shown) may be used to by a patient on himself/herself by directing the targeting mechanism onto his/her retina.

### Handle Portion

Referring back to FIGS. 1 and 2, the handle portion 160 contains the bulk of the electronics, as well as the activation switch 180 and the power supply 170. As described previously above, the handle portion 160 may also include a dosage adjuster 1530a (shown in FIG. 15c) for adjusting the amount of fluid 122 that is discharged per use. The handle portion 160 includes an elongated body 162 having a top end 164, which is connected to the body portion 130, as well as a bottom end 165, which is configured for removable insertion into a base 166.

In a non-use operation, the device 100 is preferably disposed in the base 166, as shown in FIGS. 1 and 2. The base 166 typically rests on a desktop and holds the device 100 such that the device 100 can simply be lifted from the receiver for use. The base 166 includes a cavity 167 that is sized and shaped to securely receive the bottom end 165 of the handle portion 160. The base 166 may also be weighted to keep the device 100 from toppling over after the device 100 is inserted into the base 166. Alternately, the base 166 may include an adhesion device, such as a suction cup or an adhesive (not shown), to keep the device 100 from toppling over.

Preferably, the handle portion 160 and the base 166 may be separately machined from solid metal or plastic stock, or may be injection molded with impact resistant polymer resins, such as abs, polycarbonate, pvc, or other suitable material, as will be recognized by those skilled in the art. The handle portion 160 may optionally include a rubberized grip 168, at least along a length of the handle portion 160 facing the distal end of the device 100. The rubberized grip 168 is softer for the user and helps prevent the user from accidentally dropping the device 100. The grip 168 may also include indentations for a user's fingers to enhance ergonomics. The grip 168 may be manufactured from a material having a hardness in the range of 10-50 Shore A that may be molded separately and bonded onto the handle portion 160. The material of the grip 168 may also be overmolded.

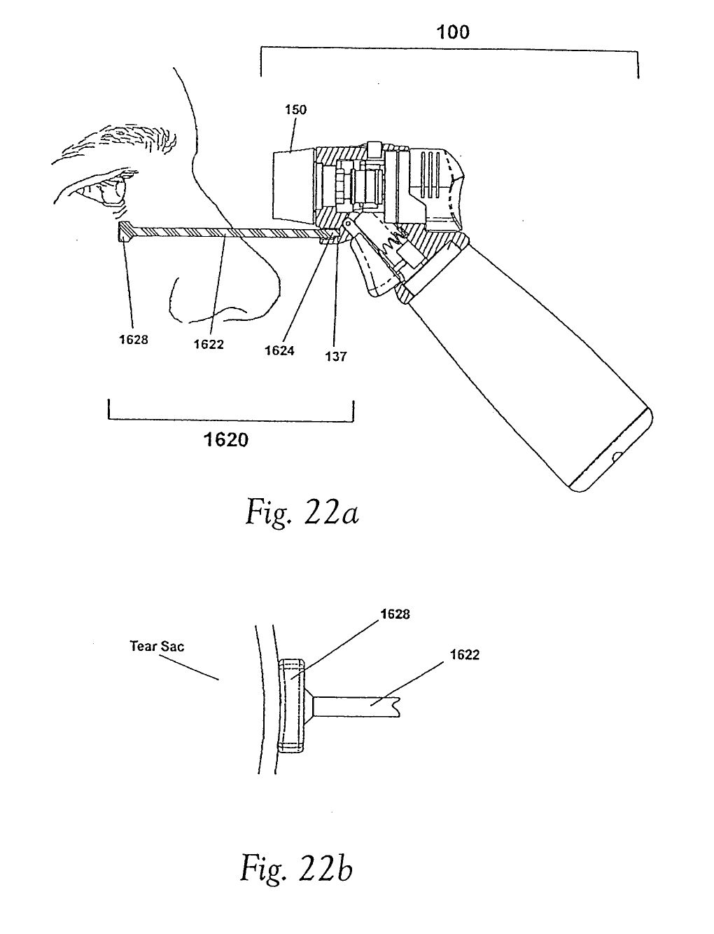

Referring now to FIGS. 22a and 22b, an optional mechanical targeting means 1620, for setting an optimum distance between the nozzle assembly 150 and the patient's eye, is shown. In lieu of the electronic targeting means 1540 shown and described with respect to FIGS. 14 and 17a-21c, the targeting means 1620 may be mechanically incorporated into the device 100.

The targeting means 1620 includes a generally elongated member 1622 that includes a connected end 1624 that is releasably inserted into the spacer recess 137, and a free end 1628 that is disposed away from the connected end 1624. As shown in FIG. 22b, the free end 1628 is generally “tee-shaped” and is preferably formed in the shape of an eyelid depressor to depress the tear sac under the eye and to provide a larger ocular surface area for contact with the fluid 122 being dispensed from the device 100. Since the free end 1628 engages the patient and the patient's eye area, it is preferred that the targeting means 1620 is disposable between uses to avoid any contamination from one patient to the next.

Preferably, the elongated member 1622 is constructed from impact resistant polymer resins, such as abs, polycarbonate, pvc, or some other suitable rigid material to minimize deflection of the elongated member 1622 during operation. Also preferably, the free end 1628 is either coated with or constructed from a soft material, such as rubber in order to reduce the likelihood of eye injury in the event that the free end 1628 accidentally engages the eye.

### Power

A preferred power source 170 for the device 100 is battery power. As can be seen in FIGS. 1 and 2, a battery 172 is removably inserted into the bottom end 165 of the handle portion 160. A cover 169 retains the battery 172 in the handle portion 160. The cover 169 is removable so that the battery 172 may be easily replaced. The cover 169 may be releasably connected to the handle portion 160 by clips, threaded fasteners, or other means known to those skilled in the art.

The battery 172 may be a single-use lithium ion or alkaline type, or the battery 172 may be rechargeable lithium-ion, nickel-cadmium, nickel-metal-hydride, or other battery type. The battery 172 may be a single battery or a plurality of batteries electrically connected in series. For example, two lithium photo batteries, NEDA/ANSI type CR2 (e.g. Duracell Ultra CR2 LiMnO²) may be connected in series to power the device 100. The batteries 172 are preferably rated for 3v and approximately 2000 mAh. The batteries may also include single or double AA and AAA cells that are rated for 1.2-1.5V and have lower overall capacity. The batteries 172 are connected in series to provide a total capacity 2000 mAh at 6v. The batteries 172 preferably have a peak current rating of at least 1.8 a.

If a rechargeable battery is used, a charger is required. Those skilled in the art will recognize that the charger may be integrated into the device 100 or enclosed in a separate enclosure, such as in the base 166. The base 166 includes a standard 110v electrical cable 1610 extending therefrom that is electrically connected to an ac/dc converter (not shown) in the base 166 that converts 110v ac supply to 6v dc. The base 166 also includes a pair of contacts (not shown) that engage recharger contacts (not shown) in the bottom end 165 of the handle portion 160 when the device 100 is inserted into the base 166.

Alternatively, the device 100 may be designed such that the battery 172 can be easily removed from the device 100 and charged in a separate charger (not shown). A further alternative is to replace the battery with an ac-to-dc converter, and power the device 100 through a line cord connected to an ac source.

### Activation Switch

An activation switch 180 extends through the handle portion 160 to activate the device 100 upon a user engaging the activation switch 180. The activation switch 180 is preferably a button, as is shown in FIG. 2, or some other suitable device, such as a trigger, as will be recognized by those skilled in the art. Alternatively, the activation switch may be a foot switch (not shown) that is electronically connected to the system controller 190 to activate the device 100, such as by an electrical line.

The activation switch 180 is electronically connected to the system controller 190 via leads 182, 184. Preferably, the activation switch 180 is a three-position switch such that, when the activation switch 180 is depressed an initial amount from an open position to an initially closed position, the device 100 is activated. This activation illuminates the activation indicator 1310 to indicate that the device 100 is about to operate. When the activation switch 180 is completely depressed, the activation switch 180 transmits a signal, through the system controller 190, to operate the prime mover 140 for a period of time determined, through the system controller 190, by the settings on the dosage adjuster 1530. Preferably, the time period for operation extends between approximately 0.5 and 5 seconds. However, operation time of the prime mover 140 is not dependent on the duration of time that the activation switch 180 is depressed, but on the settings of the dosage adjuster 1530. However, it is preferred that, if the activation switch 180 is depressed for an extended period of time, such as greater than two seconds, the system controller 190 interprets the signal received from the activation switch 180 as a signal to run the device 100 continuously for a predetermined, extended period of time, such as thirty (30) seconds, such as to run a cleaning solution such as saline, through the device 100 to clean the device 100. Alternatively, if the activation switch 180 is depressed for longer than the predetermined period of time, the system controller 190 will provide power for the prime mover 140 to operate as long as the activation switch 180 is fully depressed.

### Electronics

The primary function of the system controller 190 is to energize the prime mover 140, which is preferably a piezoelectric transducer assembly or other piezo device, as described above. When energized, the prime mover 140 generates a mist of fluid droplets from the fluid 122. The energizing signal for the prime mover 140 must excite the prime mover 140 at the proper resonant frequency, and must supply enough energy to the prime mover 140 to cause misting. A simple user interface, such as the activation switch 180, is required for operation and control of the prime mover 140. A microprocessor 192 will be used to provide intelligence for the interface between the activation switch 180 and the prime mover 140, and to supervise the circuits driving the prime mover 140, as well as all of the electronic features.

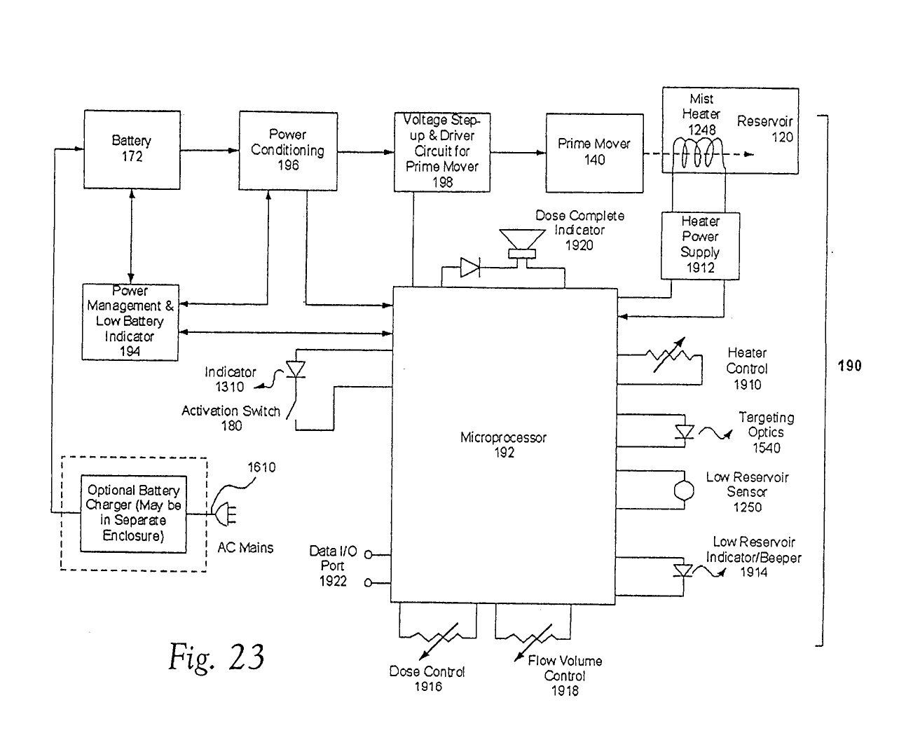

The system controller 190 controls operation of the device 100 and includes a microprocessor 192, preferably in the form of a PCBA (printed circuit board assembly), to incorporate of the electronics for operation of the device 100. FIG. 23 shows an electronic block diagram for a preferred embodiment of the system controller 190. The microprocessor 192 is housed in the system controller 190, through which a majority of the operation of the device 100 passes. The system controller 190 preferably also contains a non-volatile memory, input/output (“i/o”) devices, digital-to-analog (“d/a”) and analog-to-digital (“aid”) converters, driver circuits, firmware, and other electronic components, as will be described in detail herein. Alternatively, those skilled in the art will recognize that simple logic components may be used.

The activation switch 180 is part of a normally open (“NO”) circuit that includes the activation indicator 1310. As described above, the activation switch 180 is a three-position switch, with the first position in the NO condition. The second position, when the activation switch 180 is depressed part way, powers the activation indicator 1310 to indicate to the user that the device 100 is on. The third position, when the activation switch 180 is fully depressed, activates the device 100 to operate the prime mover 140 to generate a mist from the nozzle assembly 150 for medication dispensing to the patient. To conserve power and lengthen operational battery life, all circuits are disconnected from power while the activation switch 180 is open.

A power management & low battery indicator 194 includes an electronic circuit that automatically measures the battery voltage and provides a visual or audible (beeping) indication if the voltage has dropped below a preset level. Power management chips (also known as “gas gages”) are commercially available for various battery types, or such a circuit may be constructed from discrete components. Preferably, the circuit also provides “sleep” or “hibernate” modes, as are known to those skilled in the art, in which battery life is extended by reducing power consumption when the device 100 has been inactive for a preset amount of time.

An optional power conditioning circuit 196 provides a constant and regulated voltage to the rest of the system controller 190. Power conditioning chips are commercially available for various voltage and current requirements, or alternatively, such a circuit may be constructed from discrete components.

A voltage step-up & driver (vsd) circuit 198 powers the prime mover 140. For a prime mover 140 that includes the piezo device described above, the purpose of the vsd circuit 198 is to drive the piezoelectric crystal contained in the piezo device at a desired resonant frequency. Different crystals and piezoelectric assemblies have different resonant frequencies, as well as different q-factors, so the vsd circuit 198 is preferably custom designed to match the operating characteristics of the particular piezo device. The vsd circuit 198 contains an oscillator formed of integrated and/or discrete components such as power transistors, power diodes, capacitors, and coils.

Preferably, the piezo device is driven by a square wave at its resonant frequency in the range of 50 khz to 200 khz, preferably 180 khz Other waveforms are possible. Since each piezo device has a slightly different resonant frequency, the circuit will use a phase lock loop (PII) or other feedback technique with a voltage controlled oscillator (vco) to lock on to the piezo resonant frequency and to automatically adjust the drive signal frequency as the resonant frequency varies. The piezo device is preferably driven by a peak-to-peak signal in the range of 200v or less, or as appropriate to provide sufficient misting. Using the preferred Omron piezoelectric device described above, the mist volume produced with this method is significantly below approximately 10 microliters/second.

The system controller 190 also optionally includes a heater control 1910 and that is electronically connected to the optional reservoir heater 1248 to heat the fluid 122 in the reservoir 120, as desired. The heater control 1910 includes a feedback loop to control the desired temperature of the fluid 122 in the reservoir 120. A heater power supply 1912 is also electronically connected to the system controller 190 to provide a power supply to the optional heater 1248.

### Low Fluid Level

If the device 100 includes the low level sensor 1250 in the reservoir 120 as described above, the device 100 also includes a low fluid level alarm 1914 that is set to alarm when the fluid 122 in the reservoir 120 is depleted to a predetermined level. The low reservoir sensor 1250 is programmed to transmit a signal to the system controller 190 when the fluid level reaches the predetermined level. The system controller 190 in turn transmits a signal to the alarm 1914. The alarm 1914 may be a visual alarm, such as a blinking light, or the alarm 1914 may be an audible alarm, such as a beep.

### Dosage Adjustment

A manual method and apparatus for adjusting dosage amount dispensed during operation of the device 100, using the dosage adjuster 1530, 1530a has been previously described. Adjustment of the dosage adjuster 1530, 1530a transmits a signal to a dose control circuit 1916 to determine the length of time that the prime mover 140 operates to dispense the fluid 122 from the reservoir 120 to the patient. The system controller 190 also includes a flow volume control circuit 1918 that determines the volume of the fluid 122 per unit time that is dispensed through the prime mover 140. The total amount of the fluid 122 dispensed is determined by the value of the flow rate as determined by the flow volume control circuit 1918 times the length of time of operation of the prime mover 140 as determined by the dose control circuit 1916. Preferably, the flow volume control circuit 1918 is preprogrammed into the system controller 190, while the dose control circuit 1916 may be manually adjusted based on the type of medication and the dosage that the prescribing physician determines is necessary based on the patient's condition.

Alternatively, instead of manually adjusting the dosage amount, the dosage amount may be adjusted electronically, such as by external calibration of the system controller 190 to adjust operational values of the dose control circuit 1916 and the flow volume control circuit 1918 based on need.

### Dosage Complete Indicator

The system controller 190 also includes a “dosage complete” indicator 1920 that indicates when the device 100 has dispensed the prescribed amount of fluid 122 from the reservoir 120. The indicator 1920 may be may be a visual alarm, such as a blinking light, or the indicator 1920 may be an audible alarm, such as a beep. The indicator 1920 preferably is activated after a slight time delay, such as approximately 0.5 second, after the device 100 ceases to dispense the fluid 122 from the nozzle assembly 150. This delay ensures that the user does not remove the device 100 from in front of the patient's eye until all of the prescribed dose of medication has been dispensed from the device 100. Since the system controller 190 controls operation of the prime mover 140, the system controller 190 is able to calculate the desired delay time between stopping operation of the prime mover 140 and sending the signal to the indicator 1920 to indicate that the dosage is complete.

### Targeting Optics

If the optional electronic targeting mechanism 1540 is used, depressing the activation switch 180 to the first position transmits a signal to the system controller 190 to activate the targeting mechanism 1540, illuminating the light sources 1546, 1548 to project images on the patient's eye. The targeting mechanism 1540 remains activated when the activation switch 180 is depressed to the second position. When the activation switch 180 is released, signal to the system controller 190 ceases, and the targeting mechanism 1540 is deactivated by the system controller 190.

### Outside Communications

Optionally, the device 100 may include an input/output (i/o) device 1922 for transmitting information between the device 100 and an outside device, such as a personal computer, pda, or other such electronic device that is capable of displaying information transmitted from the device 100. Information that may be transmitted from the device 100 includes, but is not limited to, usage information, such as the number of times the device 100 was used, and at what times; dosage amount per application; and current and voltage draw of the device 100 during use, as well as other operational information about the device 100. Further, information may be transmitted from the outside device to the device 100. Such information may include, but is not limited to, clearance information to clear the system controller 190 memory of previous information that has already been downloaded to the outside device; operational information that allows the device 100 to be used with particular medicament reservoirs; temperature settings for the heater control 1910; and operational duration information to adjust the dose control circuit 1916 and the flow volume control circuit 1918 to adjust dosage amounts, as well as other information that may be transmitted to the system controller 190.

As shown in FIG. 2, the 100 device 1922 may include a port 1612 on the handle portion 160 for physically connecting the device 190 to the outside device, such as by a cable. The port 1612 may be a standard universal serial bus (USB) port, or some other suitable port as will be recognized by those skilled in the art. The port 1612 is electronically connected to the system controller 190 by a port cable 1614 that transmits information between the port 1612 and the system controller 190. Alternatively, the i/o device 1922 may include an infrared transmitter/receiver (not shown) that allows the device 100 to be placed near, but not physically connected to, the outside device to exchange information such as the information described above.

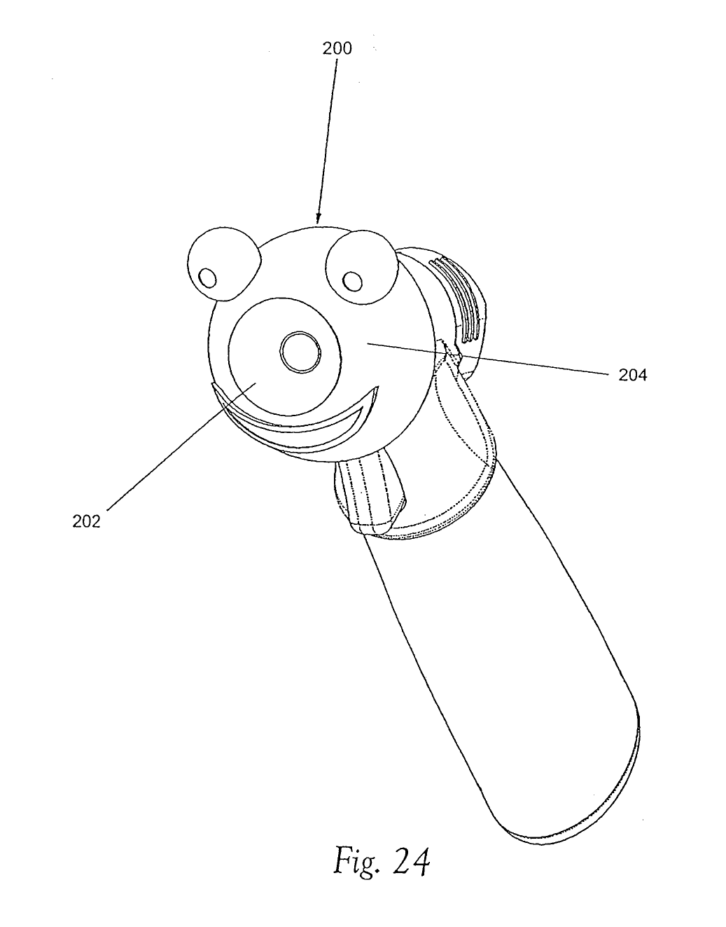

A pediatric version of a device 200 according to an alternate embodiment of the present invention, shown in FIG. 24, may include a facade 204 at the distal end 202 of the device 200 that encourages younger patients to look in the direction of the device 200. For example, for ophthalmic delivery, the facade 204 may include a clown face or an animal face that catches the attention of the patient and distracts the patient from the medicament that is being dispensed from the device 200. In the embodiment shown in FIG. 24, the nose of the facade is the mesh plate 156. Alternatively, the facade 204 may include moving parts to distract the patient during operation of the device 200.

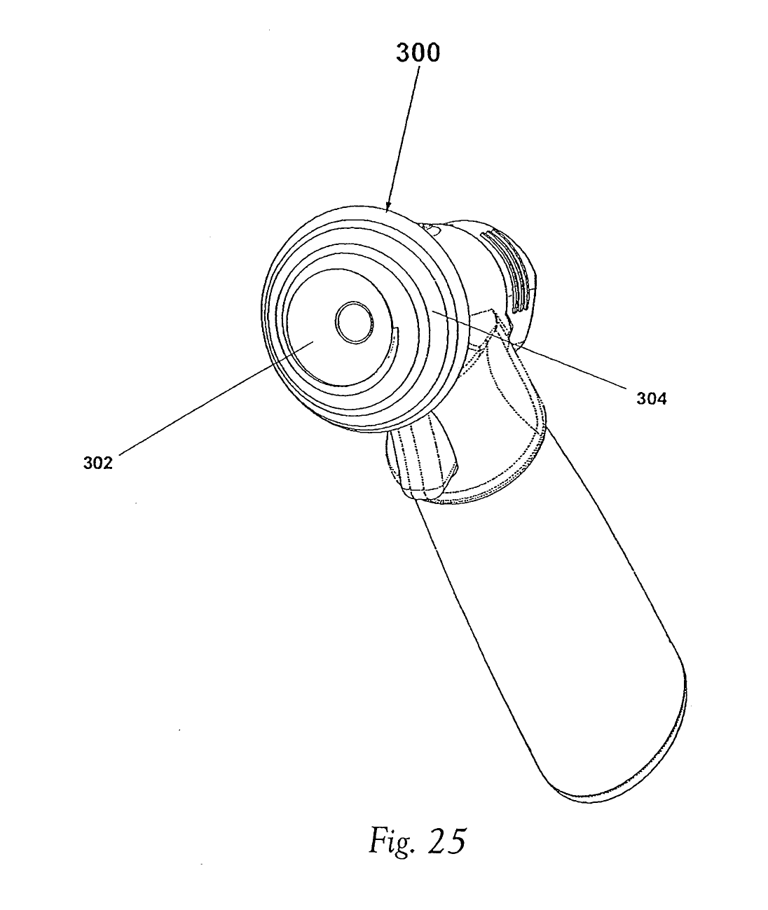

Alternatively, a veterinary version of a device 300 according to yet another alternate embodiment of the present invention, shown in FIG. 25, may include a facade 304 at the distal end 302 of the device 300 that distracts the animal that is being medicated. The facade 304 may include a moving element for the animal to focus upon during administration of the medicament.

The embodiments shown and described above may be offered in a reusable configuration. In this event, the parts may be injection molding from clear polymer resins that withstand repeated sterilization by steam autoclave, such as autoclaveable versions of acrylics, styrenes, and polycarbonates.

Alternatively, the embodiments shown may be offered as a sterile disposable. In this case it may be injection molded from a wide variety of clear polymer resins, including acrylics, styrenes, urethanes, pmma, and polycarbonates. These resins are generally compatible with industrial sterilization bye-beam, gamma, and eto.

### Use

Between uses, the device 110 is typically stored in the base 166, with the bottom end 165 of the handle portion 160 inserted into the cavity 167 in the base 166. The electrical cable 1610 is connected to an external power supply to provide electrical power to the batteries 172 to charge/recharge the batteries 172. The heater 1248, if used, heats the fluid 122 in the reservoir. The temperature of the fluid 122 is controlled by the heater controller 1910 to maintain the fluid 122 at a desired temperature.

The device 100 is designed so that it can be used by one person to self administer medicament, such as a patient in his/her home, or, the device 100 can be used by one person to administer medicament to a second person, such as a medical professional treating a patient in a medical office or a hospital setting.

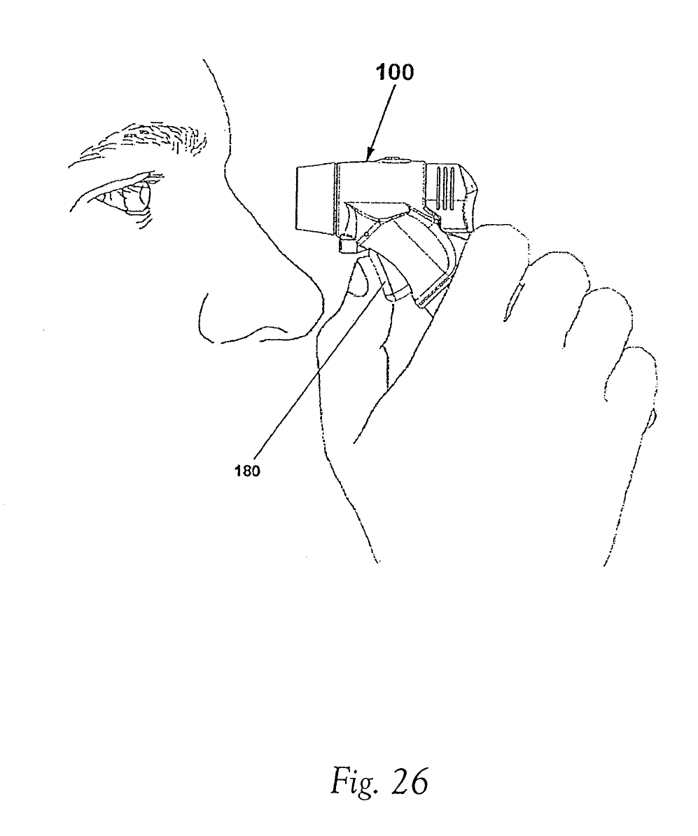

For self use, the user removes the device 100 from the base 160 and aims the discharge end of the nozzle assembly 150 toward the eye into which the user intends to insert the eye medication. If the optional mechanical targeting means 1620 is connected to the device 100, the user inserts the connected end 1624 into the spacer recess 137. The user then uses the free end 1628 of the targeting means 1620 to depress the eyelid. When the device 100 is in the desired position, the user then uses his/her thumb, as shown in FIG. 26, to depress the activation switch 180. By pressing the activation switch 180 to the first position, the activation indicator 1310 is illuminated, indicating that the device 100 is ready for operation.

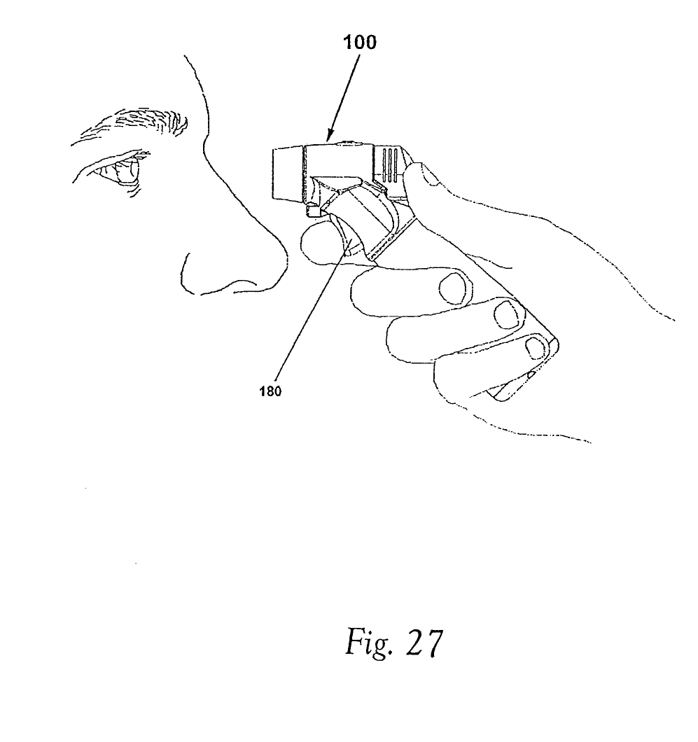

For professional use on a patient, the user, such as an optometrist or an ophthalmologist, removes the device 100 from the base 160 and aims the discharge end of the nozzle assembly 150 toward the eye into which the user intends to insert the eye medication. If the optional mechanical targeting means 1620 is connected to the device 100, the user inserts the connected end 1624 into the spacer recess 137. The user then uses the free end 1628 of the targeting means 1620 to depress the eyelid. When the device 100 is in the desired position, the user then uses his/her index finger, as shown in FIG. 27 to depress the activation switch 180. By pressing the activation switch 180 to the first position, the activation indicator 1310 is illuminated, indicating that the device 100 is ready for operation.

If the optical targeting mechanism 1540 is used, the user aims the device 100 generally toward the patient's eye and, using his/her forefinger, as shown in FIG. 27, depresses the activation switch 180 to the first position. The activation indicator 1310 is illuminated, indicating that the device 100 is ready for operation. Also, the light sources 1546, 14538 on the targeting mechanism 1540 are illuminated, projecting images onto the patient's eye. Preferably, the images are any of the images shown in FIGS. 17a-21c. The user can adjust the distance and aim of the device 100 relative to the patient's eye based on the images projected onto the patient's eye.

The remainder of the description of the operation of the device 100 is the same whether the device 100 is being used for self-administration of medication or whether the device 100 is being used by a professional to administer medication to a patient.

The user presses the activation switch 180 to the second position and then releases the activation switch 180, transmitting a signal to the system controller 190 to operate the prime mover 140. An electronic operational signal is transmitted through the power management circuit 194 and the vsd circuit 198 to the prime mover 140 which, in the case of the piezoelectric device described above, causes the piezoelectric device to vibrate, preferably at an ultrasonic frequency, along its longitudinal axis 148. The prime mover 140 is operated for a predetermined amount of time, preferably between approximately 0.5 and 2 seconds, as programmed into the system controller 190 prior to use. The prime mover 140 operates for the predetermined amount of time, regardless of how long the activation switch 180 is depressed, unless the activation switch 180 is depressed in excess of a predetermined period of time, such as 5 seconds, as will be described in more detail later herein.

The vibration of the prime mover 140 draws fluid 122 from the reservoir 120 and through the lumen 1410. The fluid 122 exits the distal end 144 of the prime mover 140 and passes through the openings 1520 in the mesh plate 156, where the fluid 122 is broken into micron-sized particles, which are directed toward the patient's eye. After the prime mover 140 has operated for the predetermined period of time, the system controller 190 ceases to transmit the operational signal and the prime mover 140 stops. At this time, the system controller 190 transmits a signal to the dose complete indicator 1920 to indicate to the user that the dosage is complete.

If the user is using the mechanical targeting means 1620, the user preferably removes the connected end 1624 from the spacer recess 137 and discards the elongated member 1622 to ensure that any bacteria from the patient's eye is not transmitted to the targeting means 1620 and then retransmitted to the next patient.

If the level of the fluid 122 in the reservoir 120 drops below a predetermined level, the low reservoir sensor 1250 transmits a signal to the system controller 190, which in turn transmits a signal to the low reservoir indicator 1914, informing the user that the reservoir 120 must be removed and a new reservoir must be inserted into the body 130.

If the low battery indicator 194 indicates that the power source 170 is at lower power, the user may insert the device 100 into the base 166 to charge the power source 170, or alternatively, replace the power source 170.

In the event that the user desires to change medication in the reservoir 120, it is recommended that the device 100 be “flushed” after removing the original medication but before using the new medication, so as not to contaminate the new medication with the old medication. In such an instance, the user inserts a reservoir containing a cleaning fluid, such as a saline solution into the body 130, and depresses the activation switch 180 in excess of a predetermined period of time, such as 5 seconds. The system controller 190 recognizes the extended depression of the activation switch 180 as the start of a cleaning cycle and operates the prime mover 140 for an extended period of time, such as for 30 seconds, or some other predetermined time, as desired. At the end of the cleaning cycle, the dose complete indicator 1920 may activate, alerting the user that the device 100 is clean, and that a new medication may now be used in the device 100.

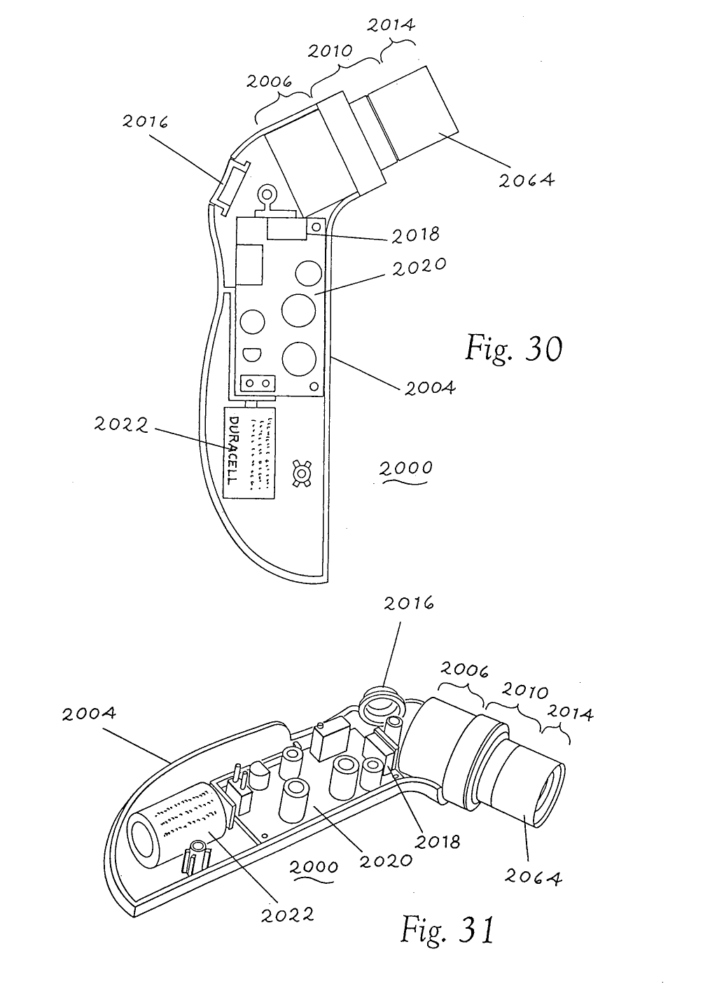

FIGS. 28-37 show another embodiment of a spray misting/atomizer device that directs a mist of drug to an eye for treatment. In particular, the spray misting/atomizer device 2000 includes a head portion 2002 and a handle portion 2004. As shown in FIGS. 30-34, the head portion 2002 preferably contains, from a proximal to a distal direction, a fluid reservoir 2006 to retain a fluid 2008, such as a drug, to be administered, a body 2010 that houses a prime mover 2012 to draw the fluid from the reservoir 2006 and propel the fluid 2008 as an aerosol out the distal end of the device 2000, and a assembly 2014 which holds a prime mover 2012 and compress any gaskets present. Within the assembly 2014 the fluid 2008 is aerosolized and a mist pattern of the fluid 2008 is formed as the fluid 2008 is directed toward its target. Note that the body 2010 and the reservoir 2006 may be just part of a blow-molded plastic eye dropper bottle. In addition, a cap may be used to cover assembly 2014 so as to protect the prime mover 2012 when not in use.

In operation, a user holds the device 2000 and, by operating an activation switch, such as button 2016, causes the device 2000 to generate a mist of the liquid, which is discharged from the head portion 2002 of the device 2000. The user simply aims an opening formed in the head portion 2002 of the device 2000 at the target eye to allow the mist to contact the eye. While the button 2016 is actuated by the thumb of the hand holding the device 2000, the activation switch can be moved to a front side of the handle portion 2004 and be in the form of a trigger so that it can be activated by a forefinger of the hand holding the handle portion 2004.

As shown in FIG. 30, a wire from the button 2016 is in communication with a microprocessor (not shown) which is attached underneath the PC board 2020 shown in FIG. 1. The microprocessor controls various operational aspects of the device 2000. The microprocessor is powered by a power source, such as battery 2022. The PC board 2020 is attached to the handle portion 2004 via screws (not shown). Note that a switch component under the button 2016 is not shown. The switch is normally open, placed between the battery and the PC board. When the switch is closed, the board is powered and producing mist. The duration of mist depends on the duration of switch closure. Preferably, the switch state is monitored by the microprocessor. When the switch is closed, the microprocessor drives a pulsed output of mist with pre-programmed timing. The duration of the switch closure is ignored, so that the output is the same regardless of input

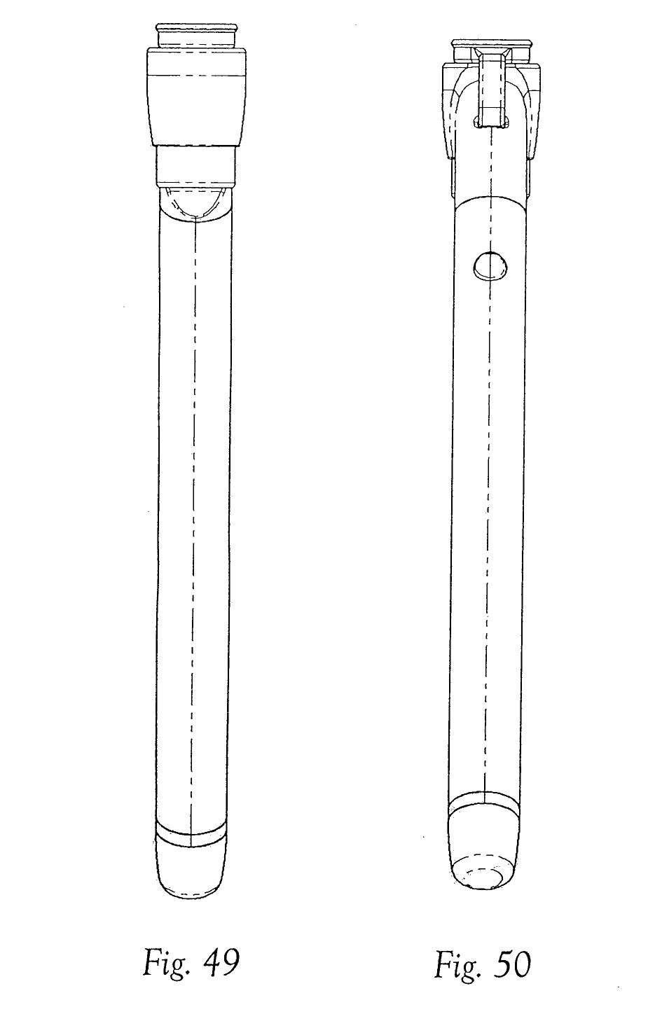

The head portion 2002 includes the body 2010 that connects the reservoir 2006, the prime mover 2012, and the assembly 2014 together. As shown in FIGS. 31-34, the head portion 2002 includes a medule bottle or container 2024, preferably made of plastic, most preferably high density polyethylene (HDPE). The bottom portion 2026 of the container 2024 defines the reservoir 2006 that contains the fluid 2008. The container 2024 also includes a neck portion 2028 that includes an exterior helical thread 2030 and a stop 2032. The neck portion defines a cylindrical channel 2034 that receives a cylindrical-like plug 2036. In particular, male member 2038 in the shape of a ring snap fits into an interior groove 2040 so that ring-shaped stop 2042 rests on shoulder 2044 of neck portion 2028. The engagement between the plug 2036 and neck portion 2028 ensures that the fluid 2008 is sealed within the container 2024. While the plug 2036 shown in FIG. 34 partially encloses wick 2046, it can also extend the entire length of the wick 2046 as shown in FIG. 49b, with optional cutouts (not shown) to allow fluid to contact the wick.

As shown in FIGS. 34-36, the fluid 2008 in the bottom portion 2026 is in fluid communication with the ambient atmosphere via a 0.124 to 0.250 inch diameter plastic fiber based wick 2046. The wick 2046 regulates fluid delivery to the mesh plate 2056 so the delivery rate is not affected by orientation. The wick 2046 can deliver the fluid in a range of 3.2 to 2.6 microliters per second. Various materials may be used to form the fibers, including polyethylene terephthalate (PET), Nylon material and polyethylene (PE). In particular, a bicomponent wick with a polyester core and a polyethylene sheath with polyethylene fibers is preferred. Softer fiber-based wick materials may perform better due to the compliance of the fiber wick which does not dampen the oscillations of the discharge plate/mesh plate 2056 as much as harder, less compliant materials. Softer wicks enable more assembly preload against the mesh plate 2056, reducing tolerance sensitivity and increasing reliability and repeatability.

In addition, non-fiber based plastic porous wicks such as those produced by Porex may be used for the wick. Small particles of plastic are heated and compressed to form a porous material. This is used extensively in markers and nibs for pens.

The wick 2046 delivers the fluid 2008 to the proximal side of the prime mover assembly 2012 via capillary action. The position of the wick 2046 is fixed via barbs 2048 formed in the interior surface of the plug 2036 that engage the wick 2046. The lip 2050 of the plug 2036 facilitates automated assembly of the wick 2046 and insertion of the wick 2046 within container 2024. The lip 2050 facilitates manual assembly and disassembly of the wick from the container. For automated assembly of the wick 2046 to the plug 2036, the extra lip may enable a yoke or other means to restrain motion of the plug along its rotational axis during insertion of the wick. Not described here, but just as essential for automated assembly is the lead-in chamfer near the barbs, guiding and slightly compressing the wick as it is inserted from the bottom. Not shown in these illustrations is an alternate design that allows the wick to be inserted from the top, into a cylindrical receptacle with slits or other openings to allow the fluid to reach the wick. A hard stop at the bottom of the receptacle would control the position of the wick better than barbs. It would also facilitate filling by allowing the wick to be inserted into the assembly last.

As shown in FIG. 34, the wick 2046 is positioned so that a small gap is formed between the bottom portion 2052 of the wick 2046 and the bottom of the bottom portion 2026. The gap aids in minimizing issues with poor bottle tolerances.

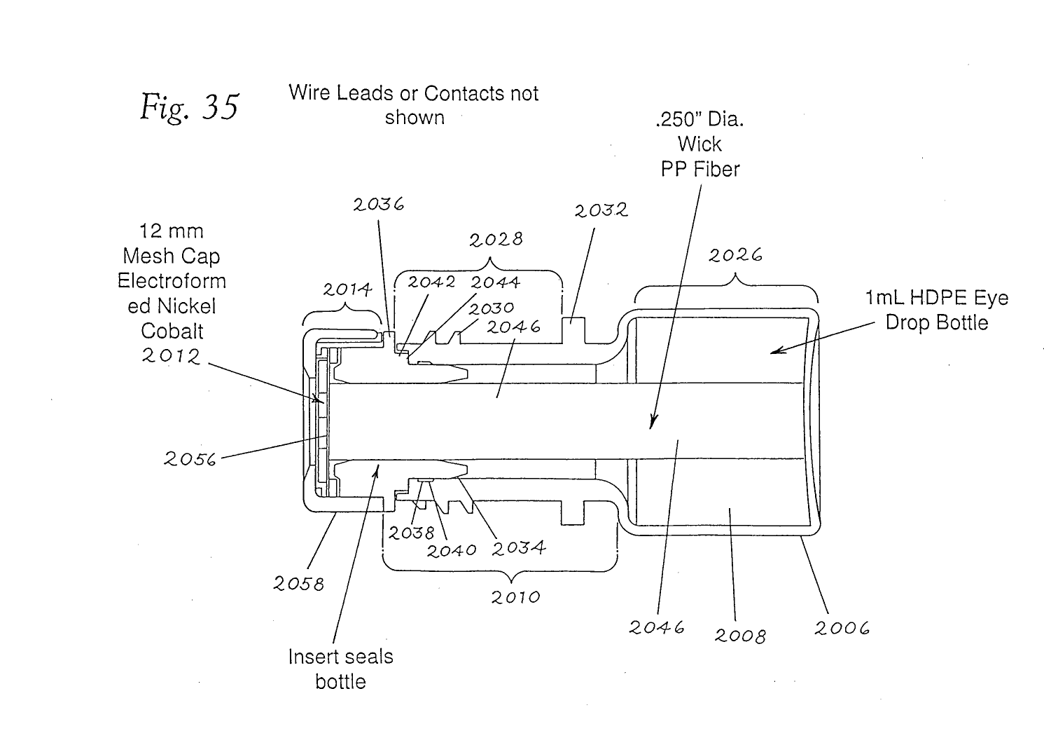

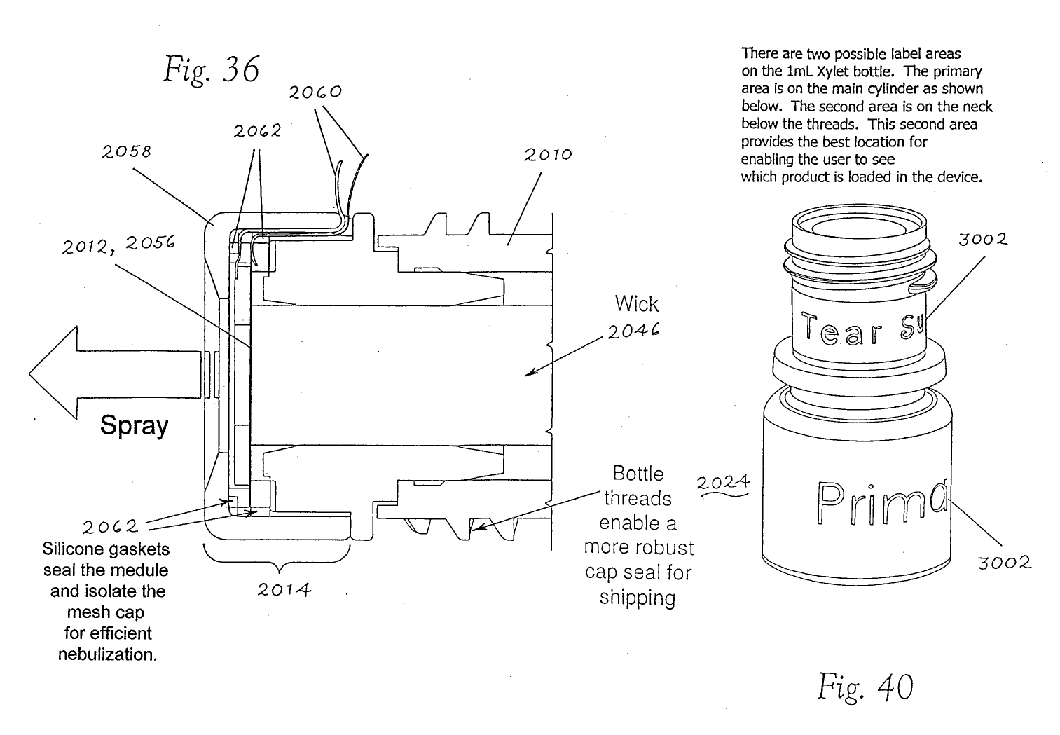

As shown in FIGS. 35 and 36, in one embodiment the wick 2046 and the cone-shaped top 2054 are enclosed by a discharge plate, such as a 12 mm diameter piezoelectric mesh plate 2056 that is electroformed of nickel cobalt. In an alternate embodiment, the plate may be formed from stainless steel sheet with laser drilled holes. An oscillator, such as the mesh plate 2056, is supported by a cap housing 2058 that is secured to the plug 2036 via small set screws (not shown), or by a snap feature, or by other means. Gaskets 2062 support the mesh plate 2056 and prevent leakage of the fluid 2008. The plate 2056 may be a stainless steel, flat plate with multiple holes, with an annular piezo transducer bonded to the distal side, wherein the holes taper from wide to narrow at the distal side.

Mesh plate 2056 may have a structure similar to that of mesh plate 156 described previously. One issue when designing mesh plates 156, 2056 is to have them operate efficiently for a range of viscosities of the fluid. This is accomplished by preparing a set of plates, wherein each plate has openings configured to handle a particular viscosity range. So, when a fluid of a certain viscosity is to be used, the user can select a particular mesh plate for that viscosity to be used in the device 2000. The openings are chosen so that it is reduced in size going from the proximal side of the mesh plate to the distal side of the mesh plate. For example, a cone or trumpet shape can be used. The openings are round or approximately round in shape. Other shapes for the openings are possible as long as the emitted fluid particles are spherical in shape. Note that while it is believed that the magnitude of the area of the opening is a key factor in determining the size of the particles emitted, other factors, such as viscosity (and temperature, which affects viscosity) and surface energy, may affect the particle size. For example, lower viscosity fluids nebulize more easily, and liquids with lower surface energy may form smaller diameter particles. With the above discussion in mind, For particle sizes that range from 5-10 microns, an approximately round opening and a diameter of 8 microns is preferred. In the case of the mesh plate being in the shape of a cymbal, a 2 micron diameter hole produces a maximum particle size of 2 microns, a 4 micron diameter hole produces a maximum particle size of 4 microns and 10 micron diameter hole produces a maximum particle size of 8.5 microns. See the article “A new cymbal shaped high power microactuator for nebulizer application,” by S.C. Shen. Note that the above described mesh plates 156, 2056 may have a structure that may promote occlusion of the openings of the plate and/or formation of a biofilm. Such effects can be minimized by employing a specific maintenance program which entails 1) capping the device 2000 which prevents deposition of airborne bacteria on the surface of the mesh plate and also prevents evaporation of the fluid in the openings of the mesh plate, 2) placing preservatives in the fluid so that bacteria are killed in the fluid and so that bacteria are killed that land on areas of the mesh plate that contain the fluid, 3) placing an ion coating on the mesh plate that attacks bacteria landing on the mesh plate, 4) placing limitations on the amount of usage of the device 2000 and 5) cleaning the mesh plate by immersing the plate in alcohol, vinegar and/or boiling water or by applying ultrasonic or ultraviolet energy to the plate.

As shown in FIG. 36 wire leads 2060 connected to PCB 2020 are led to mesh plate 2056. In the alternative, spring contacts can be used instead of the wire leads. Spring contacts would enable easy removal and replacement of the assembly. Silicone gaskets 2062 seal the cap housing 2058 so as to isolate the mesh plate 2056 for efficient nebulization. A cap 2064 with a circular opening can then be screwed onto the thread 2030 of neck portion 2028 by making a quarter turn so that the cap housing 2058, plug 2036 and container 2024 are enclosed within the cap 2064.

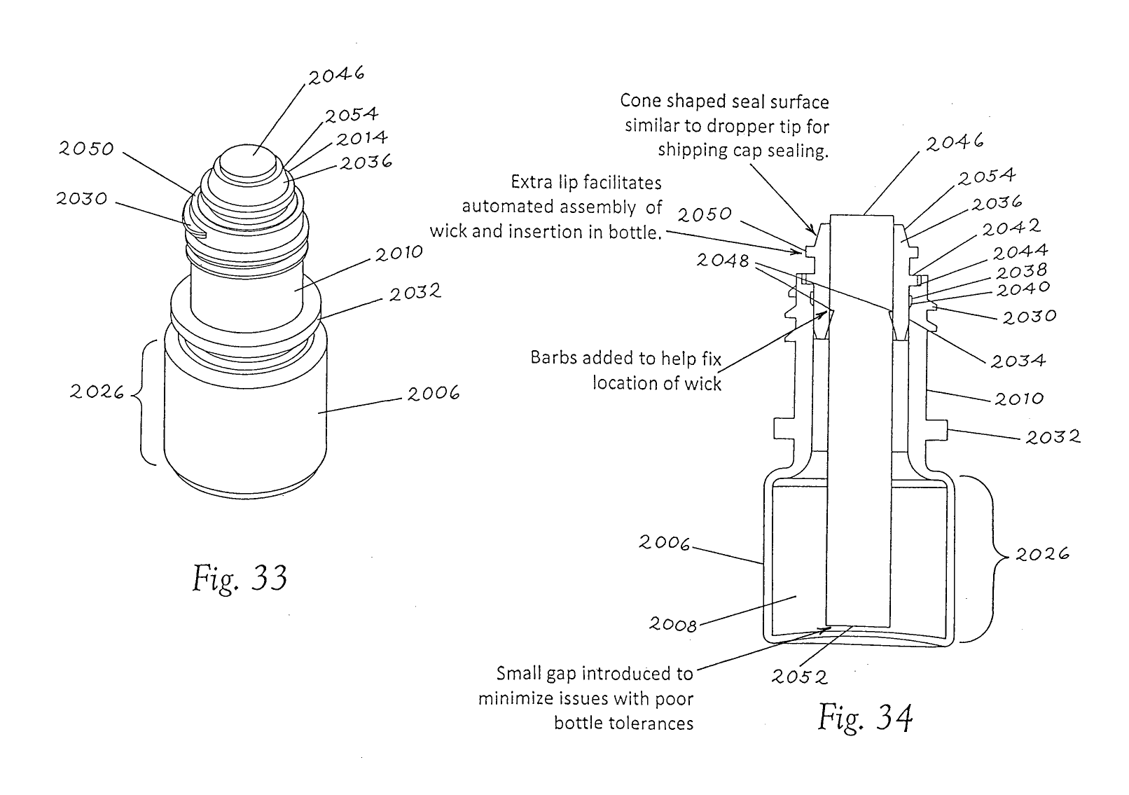

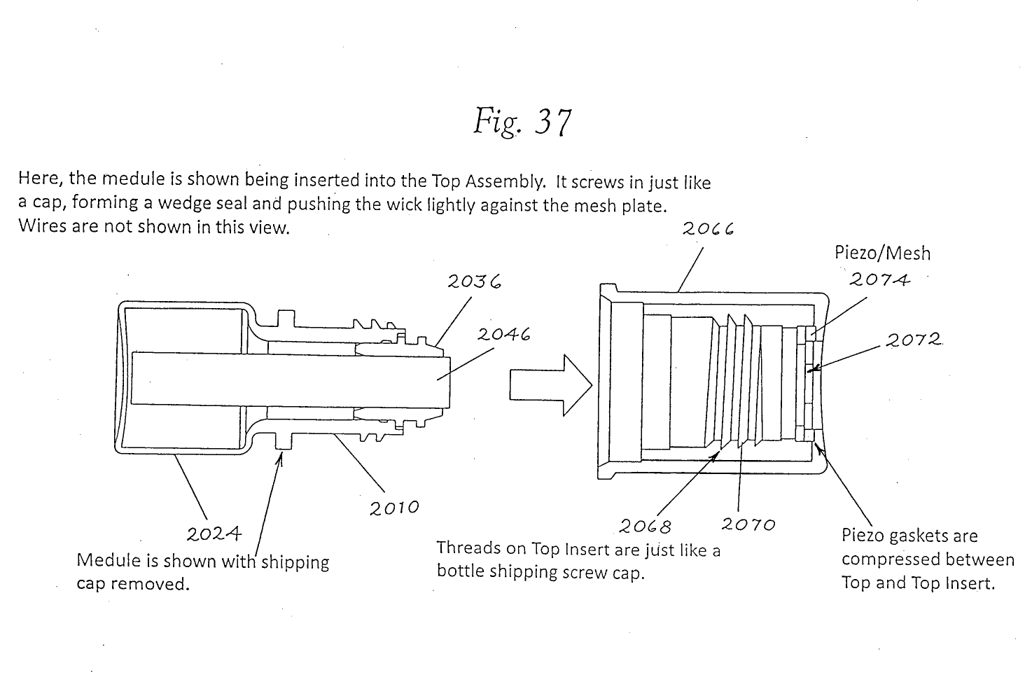

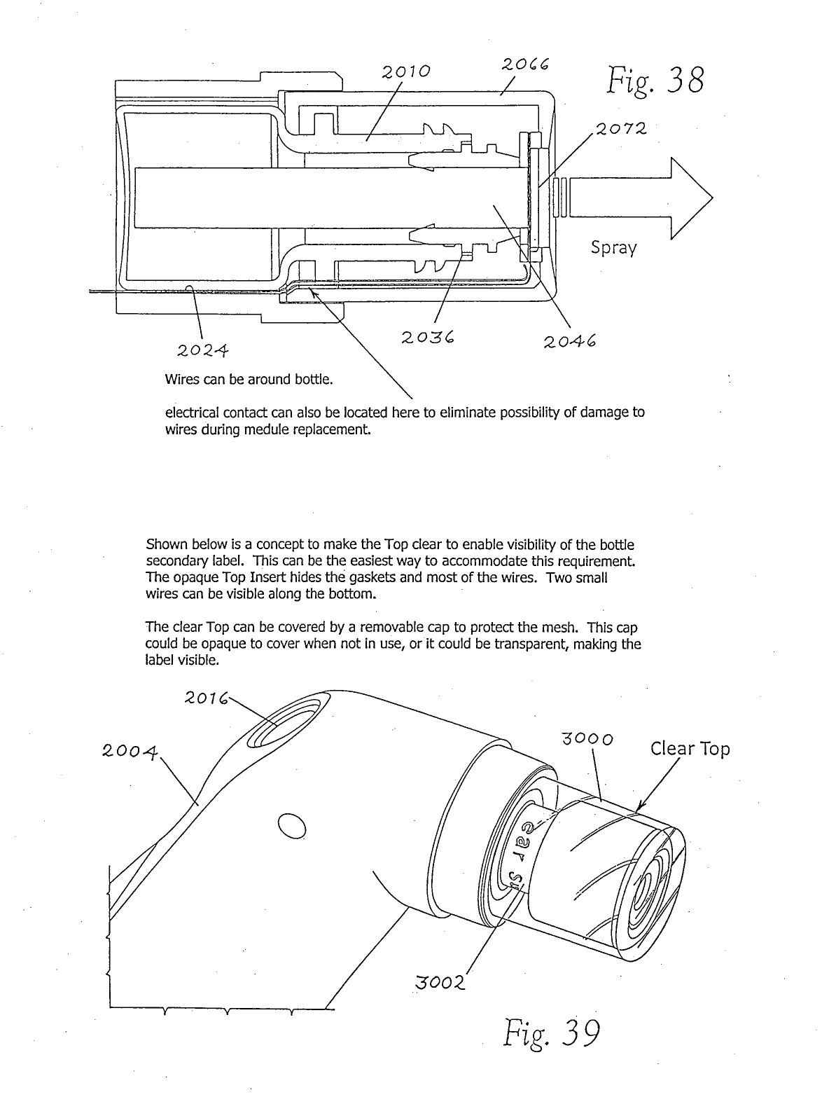

In an alternative embodiment of a spray misting device 2065, the container 2024, plug 2036 and wick 2046 of FIGS. 33-34 cooperate with a cylindrical-like cap 2066 in the manner shown in FIGS. 37-38. The container 2024, plug 2036 and wick 2046 operate in a manner similar to the container 2024, plug 2036 and wick 2046 of FIGS. 33-34. As shown in FIG. 37, the cap 2066 includes an insert 2068 having a thread 2070. A piezo mesh plate 2072 is attached to a top portion 2074 and is aligned with a circular opening of the cap 2066. Silicone gaskets 2062 isolate the piezo mesh plate 2072 for efficient nebulization. They also prevent leakage of fluid 2008 around the mesh cap 2072. A cone shaped protrusion of plug 2036 interfaces with a cone shaped receptacle of insert 2068 to create a wedge seal. This alternate embodiment enables a low cost consumable medule with a reusable piezo mesh plate 2072 assembly. The mesh plate 2072 is formed in a manner similar to mesh plate 2056 in that it is a rigid material that is biocompatible and non-oxidizing. The mesh plate 2072 has an array of openings in a manner similar to that shown in FIG. 10. The number, density, size, and shape of the openings 1520 contribute to determining mist parameters such as volume and droplet size distribution.

Piezo gaskets 2076 are compressed between top portion 2074 and the insert 2068. The gaskets 2076 are used to prevent short circuits that could result from the wetting of the mesh plate. The structure and durometer of the gaskets 2076 are such as to minimize dampening of the vibrations of the mesh plate. The annular piezo element vibrates radially, producing reciprocating axial displacement of the thin mesh structure in the center. Soft durometer annular gaskets lightly contacting the proximal side of the mesh plate and the distal surface of the annular piezo element, are probably preferred because they would not constrain the radial motion of the annular piezo element. The insert 2068 position in the top portion 2074 is fixed by friction fit, snap feature, adhesives, or other means.

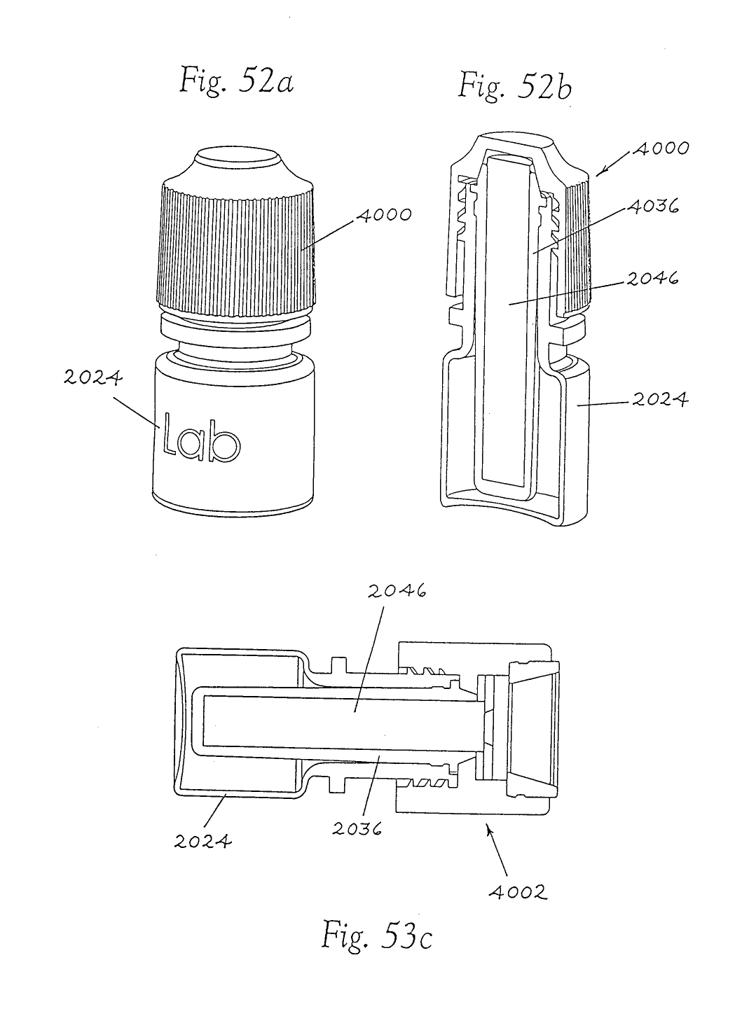

In an alternative embodiment of the spray misting device 2000 of FIGS. 28-38, the container 2024 contains the plug 4036 that extends the entire length of the wick 2046 and has cutouts (not shown) that allow the fluid in the container 2024 to be absorbed by the wick 2046 as shown in FIGS. 52a-b. A threaded bottle cap 4000 can be used to seal the wick 2046 and the liquid within bottle 2024 as shown in FIGS. 52a-b. The bottle cap 4000 can also be used to seal the wick 2046 when the plug 2036 of FIG. 34 is used. The threads of bottle cap 4000 engage thread 2030 of body 2010 so as to preserve the contents of the liquid when device 2000 is not being used. In addition, the fluid contains a preservative so as to prevent contamination of the fluid when the cap 4000 is removed.

The container 2024 and cap 4000 are designed to increase the versatility of devices 2000 of FIGS. 28-41b. In particular, container 2024 is shaped so as to be slid in and out of the head portion 2002. This modularity allows for different ophthalmic fluids to be used by the same device 2000. For example, suppose there is a patient that has two different prescriptions of ophthalmic fluids to be applied to his or her eye. In this scenario, the patient buys both ophthalmic fluids in separate containers 2024 with their corresponding caps 4000 attached thereto. The patient removes the cap 4000 from one of the containers 2024 and places the opened container into the head portion 2002 and then applies the fluid. Once finished, the patient removes the opened container 2024 and places its cap 4000 back on. The process is repeated for the other container 2024. Thus, the patient is able to apply two different fluids with the same device.

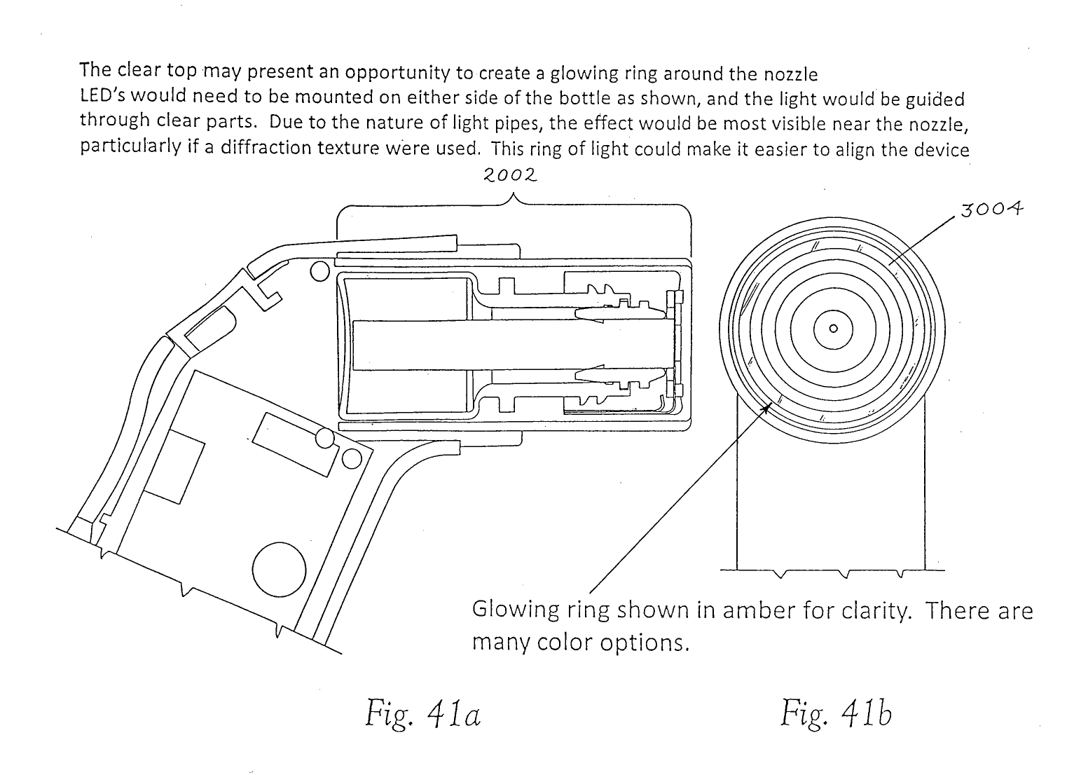

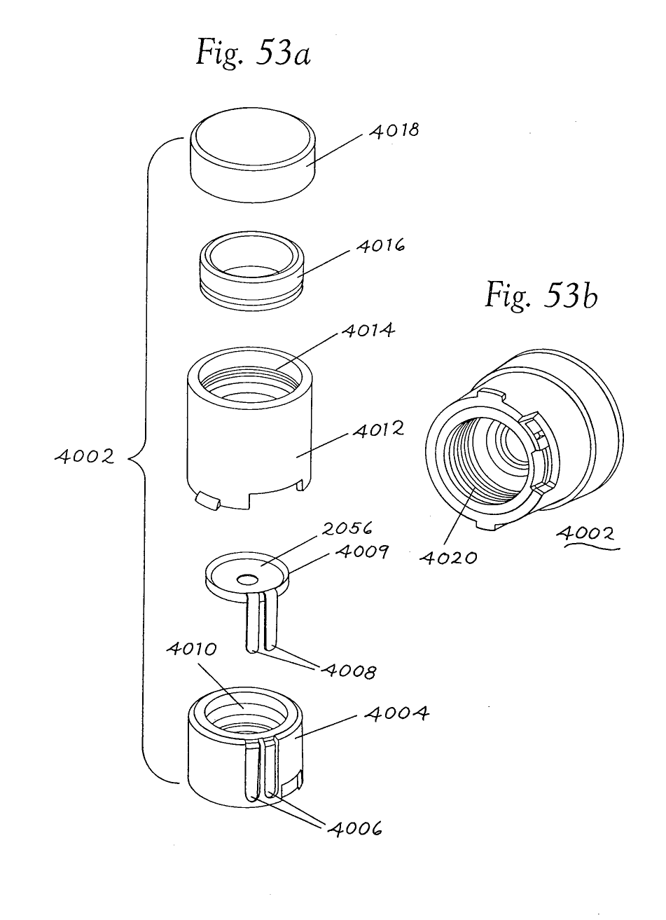

One issue regarding the above mentioned process is that when the cap 4000 is removed, the fluid is exposed to the external environment. Using a preservative in the fluid helps with reducing the risk of contamination. Another way to protect the fluid is to employ a medule top assembly that is attached to the body 2010. Once attached to the body, the medule top assembly acts as a cover that protects the wick and the fluid sent by the wick to the mesh plate. As shown in FIGS. 53a-c, the medule top assembly 4002 includes a top insert 4004 made of HDPP resin that includes two slots 4006 that receive conductor prongs 4008 electrically connected to mesh plate 2056. The prongs 4008 and mesh plate 2056 are bonded to one another by an annular piezo ring 4009. The bonding can be accomplished by soldering or silicone or elastomer encapsulation. The mesh plate 2056 is seated within a recess 4010 formed in the top of the top insert 4004. The top insert 4004 is inserted into one end of a top 4012 made of HDPP resin and snap fit within the interior of the top 4012. The top 4012 may be translucent to enhance the targeting of FIGS. 41a-b. An opposite end of the top 4012 has a recess 4014 that engages an HDPP nozzle 4016 in a snap-fit fashion. An HDPP cap 4018 can be placed over the nozzle 4016.

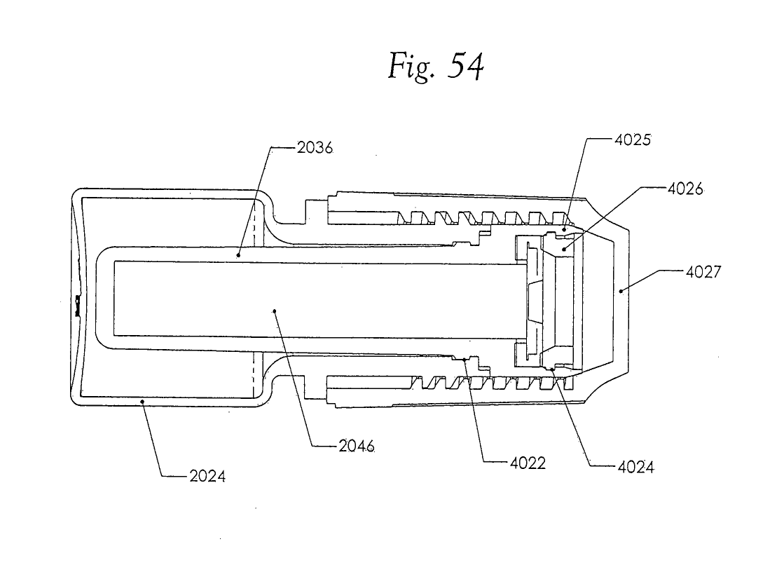

As shown in FIG. 53b, the fully assembled medule top assembly 4002 has an interior thread 4020 that engages thread 2030 of body 2010 of FIG. 34. The assembly as shown in FIG. 53c is then inserted into the spray misting device 2000, and the prongs 4008 make an electrical connection with the PCB 2020 and the misting device is operated in the manner described herein with respect to the embodiment of FIGS. 28-38. When the user is done with applying the mist to the eye, either the assembly 4002 is retained on the body (the seal between the assembly and the body 2010 being sufficient to prevent contamination) or the assembly 4002 is removed so that the container 2024 is removed and replaced by a different container 2024 with the same or a different fluid. Note that if the assembly 4002 is removed, it can be cleaned so as to remove contaminants. As mentioned previously, the removable container 2024 allows for different fluids to be loaded and dispensed by the misting device. In order to keep track which fluid is loaded in the misting device, the head portion 2002 may include a transparent window that allows the user to visualize the container 2024 and indicia on the container 2024 indicating the medication and properties of the fluid, such as concentration of the medication within the fluid. Note that when the fluid does not contain a preservative, then there likely will be a need to permanently attach the mesh plate to the container 2024 in the manner shown in FIGS. 35-36 or FIG. 54. As shown in FIG. 54, the plug 2036 has protrusions 4022 and 4024 that form snap detent and wedge seals, respectively. A mesh plate assembly is captured inside an extended lip 4025 by an annular plug 4026. A shipping cap 4027 would be removed prior to use, and the assembly shown in FIG. 54 (minus the cap 4027) would be inserted in a modified version of the spray misting device, not shown. This embodiment eliminates potential contamination of the wick surface or the proximal side of the mesh plate that may occur during medule loading.

Operation of the spray misting devices 2000 and 2065 are similar. In particular, activation of the activation switch button 2016 causes PC board 2020 to send a signal of a fixed duration that causes the mesh plate 2056, 2072 to vibrate/oscillate. The vibrating mesh interacts with the fluid drawn up by capillary action via wick 2046 so as to cause a mist in the manner described previously with respect to the embodiments of FIGS. 1-27. Of course other oscillators can be used for mesh plates 2056, 2072 as described previously with respect to the embodiments of FIGS. 1-27. For a mesh plate 2056, 2072, the preferred resonance frequency of the mesh plate is in the range of 180 kHz which requires a power of from 1-2 Watts.

It is believed that the resonance frequency is tied to the characteristics of the plume. For example, if resonance is not achieved, mesh plate displacement is not adequate to produce a plume. If resonance is damped (by overcompression of the wick for example), then the plume becomes less robust, with a lower flow rate. The vibrations of the mesh plate tend to concentrate in the center of the mesh plate, and this area seems to have the biggest displacements. Therefore, droplets are most easily produced by openings in the center of the plate, and least easily produced as you move further in radius from the center. When voltage is reduced, or the vibrations are dampened, the plume gets narrower as droplets are produced by fewer and fewer openings.

Upon pressing of button 2016, the mesh plates 2056, 2072 generate a plume of aerosol along a direction directly toward the eye, wherein the plume of aerosol travels unassisted from the discharge plate to the eye and at the eye has a momentum subliminal to at least one of an ocular blink reflex and a lacrimation reflex of the eye.

The discharged fluid has a velocity of between approximately 4 and 30 centimeters per second and a rate of fluid of between approximately 2 and 10 microliters per second is discharged. Note that activation of the button 2016 can have the atomizer operates for approximately 0.5 to 5 seconds, wherein the time of operation is independent of the amount of time that the button 2016 is pressed. Note that the flow rate can be controlled in the manner described previously with respect to the embodiments of FIGS. 1-15c. For example, the device can employ the pulse width modulation and pulse train methods described previously.

In an alternative embodiment, the button 2016 is altered so that it is able to be depressed and move along a direction parallel with a longitudinal axis of the body 2010. In this embodiment, activation of the device is accomplished by first translationally moving the button 2016 along the direction parallel with the longitudinal axis. This results in an iris diaphragm opening up and allowing the plume to leave the mesh plate. The iris diaphragm is externally positioned at an end of the head portion so that when it is in a closed position it entirely covers the mesh plate. After the diaphragm is opened, the user depresses the button 2016 which results in the generation of the plume as mentioned above. After the plume is generated, the diaphragm automatically closes. Note that other multi-positional buttons and diaphragms can be used without departing from the spirit of the invention. For example, a sliding door or a hinged door that opens when the button 2016 is translated. The sliding door and hinged door could be spring loaded.

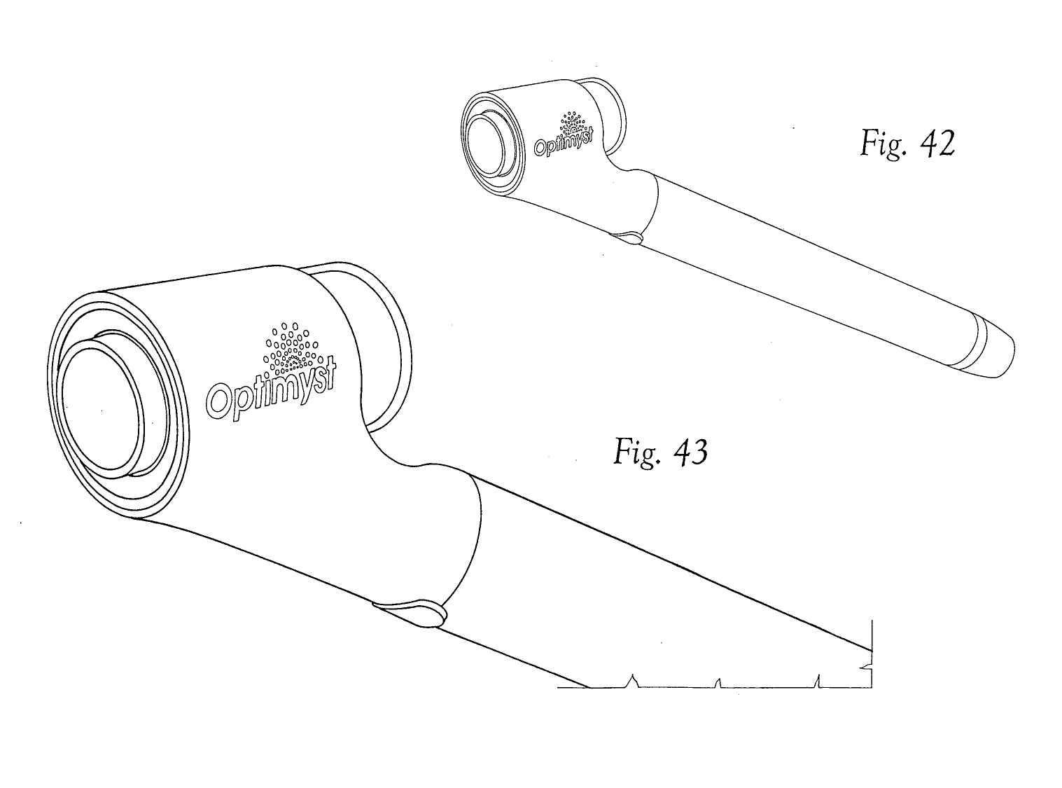

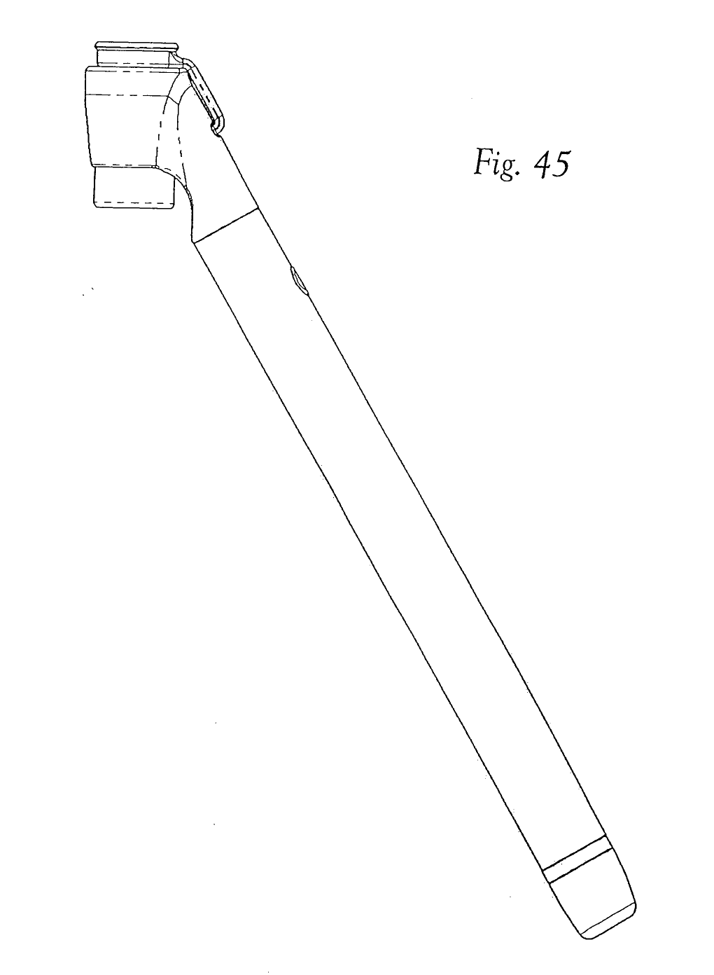

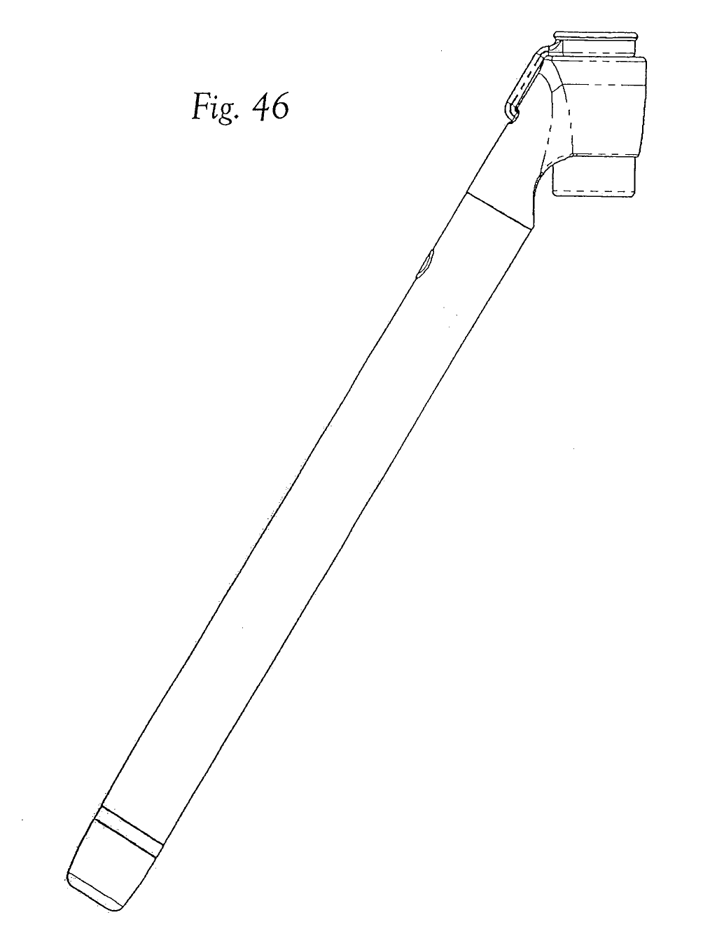

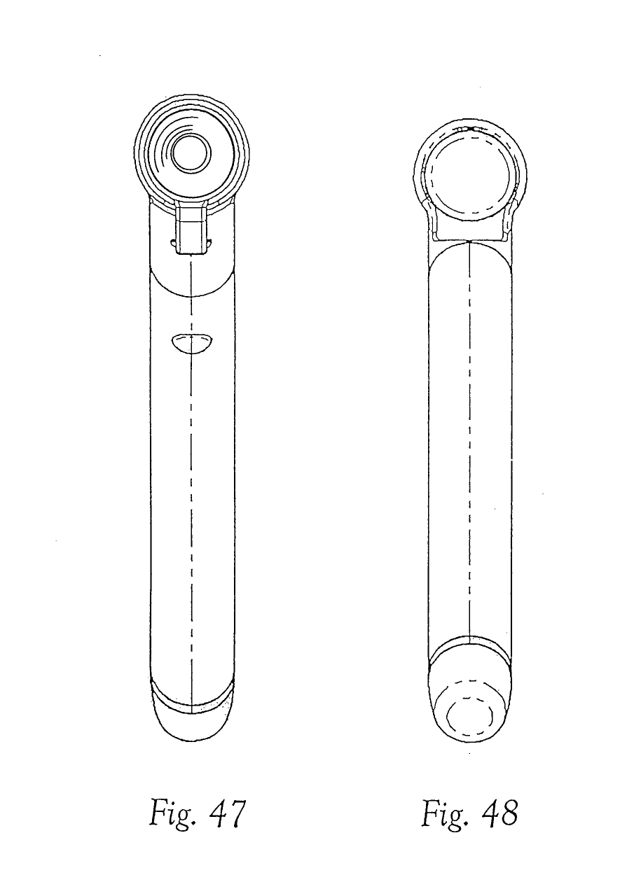

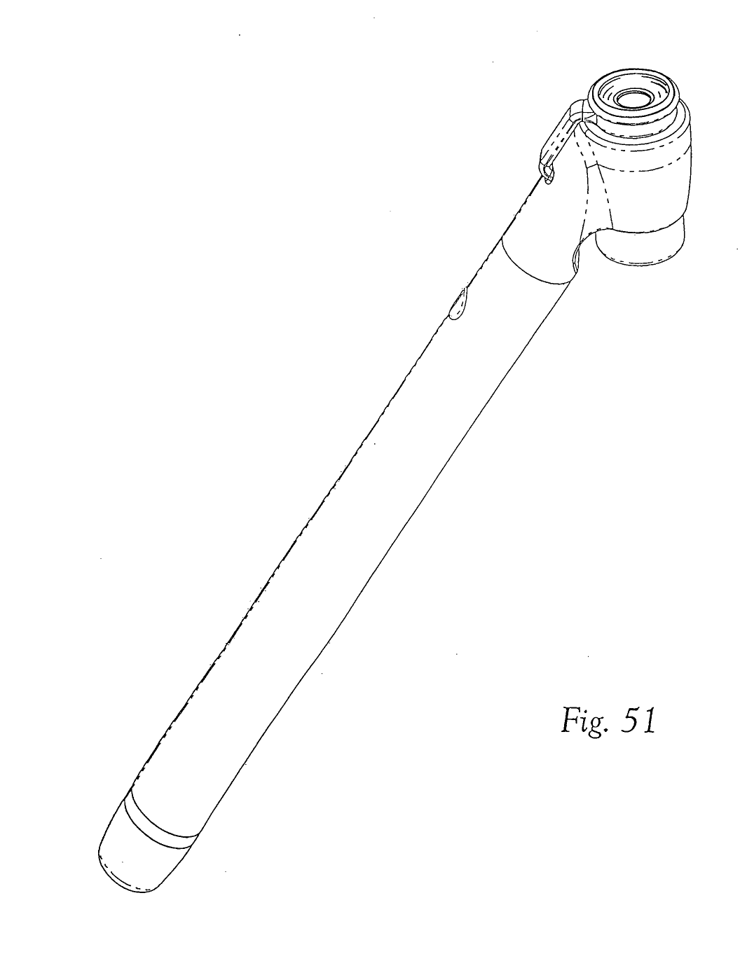

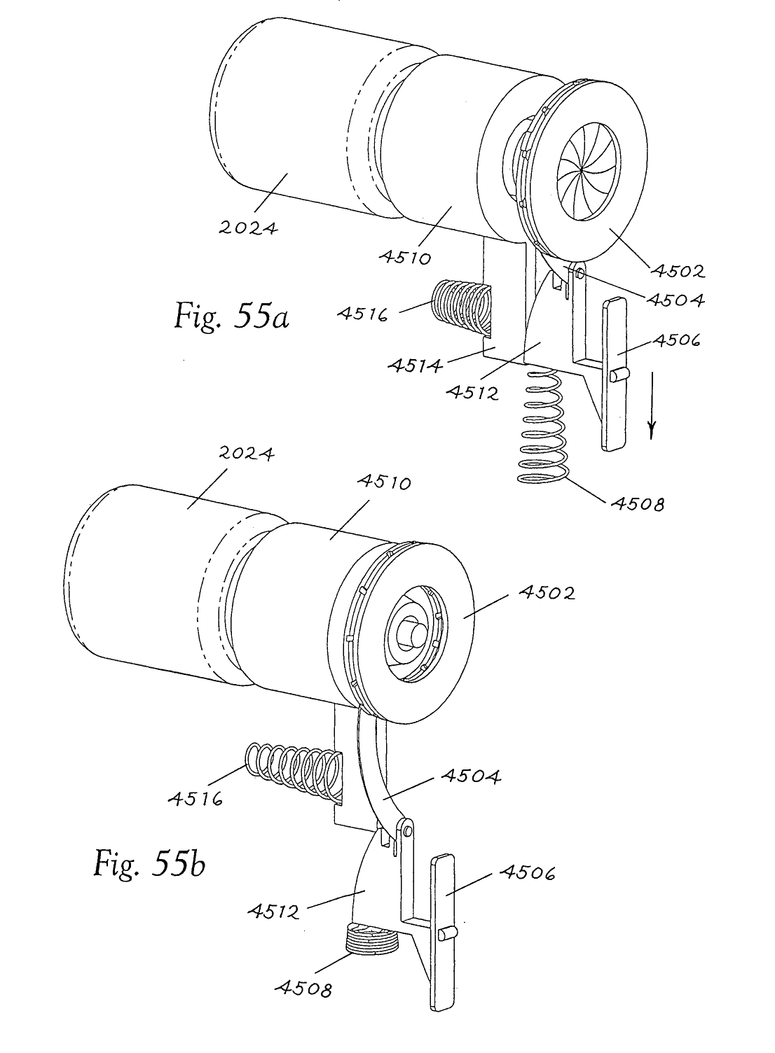

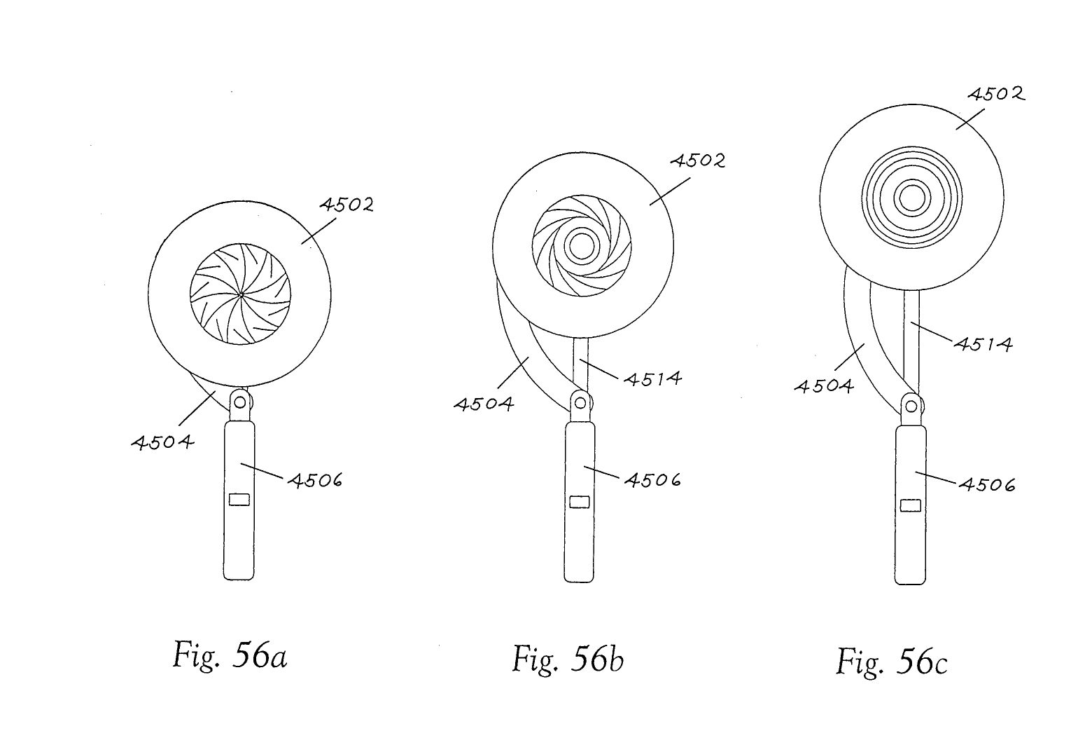

An example of an iris diaphragm that is positioned between the mesh plate and the wick is shown in FIGS. 55-56. FIG. 55a shows an embodiment of a closure system 4500 that is attached to the misting device of FIGS. 42-51 and includes a closure element, such as iris diaphragm 4502, that is coupled with a lever 4504 that is pivotably attached to a translational button 4506. The button 4506 is biased by a spring 4508 to be at a first position shown in FIG. 55a wherein the iris is in a closed position. The button 4506 is engaged at a curved portion 4512 at the first position by a ledge 4514 that is integrally attached to a shuttle mechanism 4510 that holds the container 2024, body 2010 and wick 2046. The shuttle mechanism 4510 and iris diaphragm are positioned within the head portion 5002. The shuttle mechanism is able to translate back and forth within the head portion 5002. The ledge 4514 and the shuttle mechanism 4510 are biased by the portion 4512 away from the mesh plate (not shown) attached at the distal end of the head portion 5002.

In order to open the diaphragm 4502, the button 4506 is pushed downward (see arrow FIG. 55a) which allows the ledge 4514, shuttle mechanism 4510 and wick 2046 to be moved toward the mesh plate via spring 4516. As shown in FIG. 55b, fully pushing the button downward results in the lever 4504 being pulled down resulting in the opening of the diaphragm. At the position shown in FIG. 55b, the mist can be dispensed toward the eye. Various positions of the diaphragm are shown in FIGS. 56a-c. Note that the above described embodiment can be adapted for multi-leaf diaphragms. In addition, the closure system could be adapted to act as a cap, with the mesh plate between the diaphragm and the wick. In this scenario, the medule is moved back a bit so that the wick does not protrude through the diaphragm.

As another alternative embodiment, mesh plate protection device can be employed in conjunction with the multi-positional button described previously. In this embodiment, sliding of the button 2016 would result in both an iris diaphragm opening and the mesh plate translating toward the wick. At this position, generation of the plume can be initiated. When generation of the plume is completed, the diaphragm automatically closes and the mesh plate is automatically translated away from the diaphragm and the wick. The iris diaphragm in this case is positioned between the mesh plate and the end of the wick nearest the mesh plate. The mesh plate protection device is thus able to separate the moist wick from the mesh plate during periods of disuse so that potential corrosion, biofilm formation and microbial overgrowth is avoided from contaminating the mist directed to the eye. Note that in the alternative, the mesh plate can remain stationary and the wick can be moved away from the mesh plate during closure of the diaphragm or both mesh plate and wick move away from one another. Note that other types of diaphragms can be used without departing from the spirit of the invention. In addition, the mesh plate protection device can be used in conjunction with the external iris diaphragm described previously.

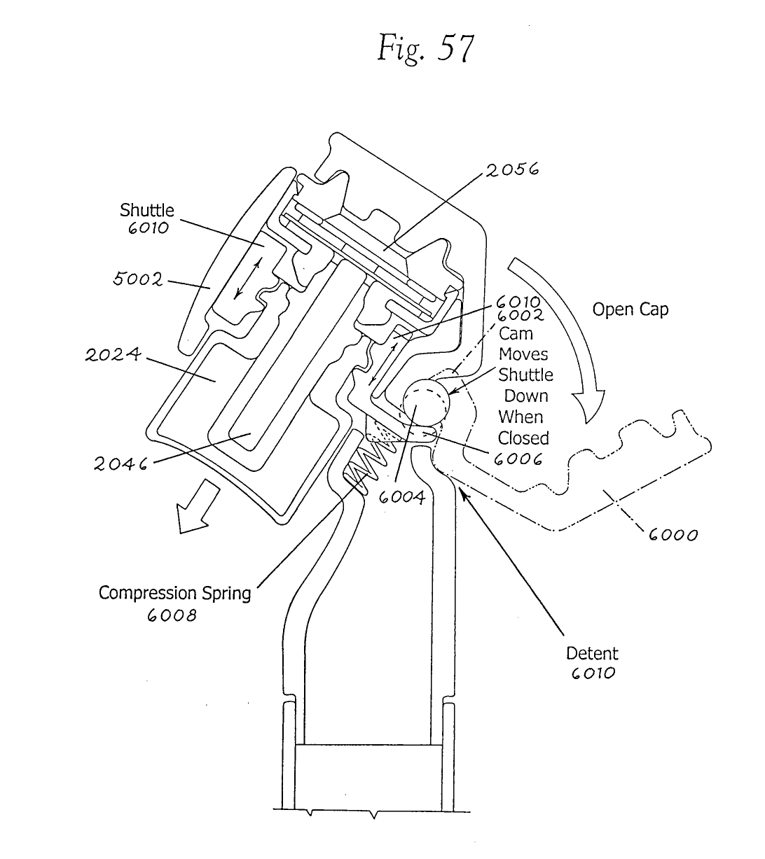

Another alternative embodiment of a mesh plate protection device that can be employed with the devices of FIGS. 1-54 is shown in FIG. 57. In FIG. 57, the protection device is used with the misting device of FIGS. 42-51. In this embodiment, a closure element, such as hinged cap 6000, moves from a closed position (misting device is inoperative) to an open position (denoted by dashed lines) wherein the misting device is ready to generate a mist. An end 6002 of the cap 6000 engages a cam 6004 that rests on a ledge 6006. When the cap 6000 is rotated to the closed position, the cam rotates causing the ledge 6006 to overcome compression spring 6008 and translationally slide away from the mesh plate 2056. The cam and spring lock the cap in the closed position. Ledge 6006 is integrally attached to an annular shuttle 6010 that translationally slides within head portion 2002 (see arrows). Movement of the shuttle 6010 causes the container 2024 and wick 2046 to move away from the mesh plate 2056 since the shuttle 6010 has threads which engage the threads of the container 2024. Thus, in the closed position, the wick 2046 is isolated from the mesh plate 2056 and the mesh plate 2056 is covered which reduces the risk of contamination. Rotating the cap 6000 to the open position allows the spring 6008 to overcome the cam 6002 and translationally move the shuttle 6010 toward the mesh plate 2056 and lock the cap 6000 against a detent 6010 formed in the handle portion of the device.

Other ways to avoid contamination of the fluid/mist directed to the eye are possible in conjunction with the mesh plate protection device. For example, a nanoscale fine coating of either ionic silver and/or polytetrafluorethylene (PTFE, sold under the trademark Teflon) can be placed on both the wick and the surface of the mesh plate to reduce microbial contamination of the fluid and retard biofilm formation, respectively. In addition, low pH (less than or equal to 2.5) formulations and chemically preserved solutions can be used in the fluid and in combination with the coating in order to prevent microbial combination. Furthermore, a UV LED can be placed near the mesh plate, wherein the LED is automatically activated after the mist has been generated in order to disinfect the mesh plate. When all of the above devices and compositions are used in combination, an effective deterrent to contamination of the mist applied to the eye is provided.

Without limiting the type of fluids that could be contained in the reservoir 2006 and dispensed by the spray misting devices 2000, 2056 of FIGS. 28-38, diagnostic agents used by the medical professional that could be delivered with the present invention include mydriatics/cycloplegics, anesthetics, flourescein and flouresceinlanesthetic combinations, and mydriatic reversal agents. Other agents which could be delivered with the present invention include over-the-counter agents, e.g., ophthalmic decongestants and lubricants, glaucoma medications (prestaglandins, beta blockers, alpha adrenergic agents, carbonic anhydrase inhibitors, miotics), and other ophthalmic medications. Optionally, several different therapeutic agents can be custom formulated in a single fluid to simplify adherence to multiple medication regimens.

Again, while an envisioned used for the devices 2000, 2056 of the present invention is directed toward ophthalmic use, those skilled in the art will recognize that the device 2000, 2056 of the present invention may be used in other areas, such as respiratory treatment, and that other fluids, including respiratory medicaments, may be contained in the reservoir 2006 instead.

After several uses of the misting device 2000, 2056, the amount of fluid 2008 can become so low that more fluid is needed. This can be accomplished by removing the medule which is considered to be the container 2024, the plug 2036 and the wick 2046. Removal of the medule is performed by twisting the cap 2064 ⅛ turn and removing it, disengaging the bayonet style mount. The medule can then be removed by pulling it out. The medule assembly shown in FIG. 35 has spring contacts in the preferred embodiment that automatically disengage when it is removed. A pre-filled replacement medule is inserted into the head portion 2002 and the cap 2064 is reattached. In an alternate embodiment, the cap 2064 is removed as described above. The medule and cap 2066 are removed by pulling them out. The medule is then disengaged from the cap by unscrewing it. A pre-filled medule is then screwed into cap 2066 and both are inserted into the head portion 2002 and the cap 2064 is reattached.

While the embodiments of the present invention described above are preferably used to deliver medicament to a patient's eye, those skilled in the art will recognize that the embodiments of the present invention may be used with a respiratory medication instead of an ophthalmic medication, and that the invention may be used in the treatment of respiratory ailments instead of ophthalmic ailments.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

One example of such a modification is shown in FIGS. 39 and 40, wherein the caps 2064 of the embodiments of FIGS. 28-38 are replaced by a partially clear or transparent cap 3000. In addition, the container 2024 includes indicia 3002 as shown in FIGS. 39-40 that are representative of the identity of the fluid 2008 contained within the container 2024. As shown in FIG. 39, the cap 3000 can include an opaque upper portion so as to hide the gaskets and most of the wires. The cap 3000 can also be entirely clear or transparent. In either case, a user can easily determine what type of fluid/medication is to be administered prior to pressing the button 2016. Note that an additional cap can be used to cover the cap 3000 so as to protect the mesh plate 2056, 2072. The above modification can be used with the embodiments of FIGS. 1-27 as well.

Another example of a possible modification of the embodiments of FIGS. 1-40 is shown in FIGS. 41a-b. In this case a light pipe 3004 is placed symmetrically around the wick 2046, the mesh plate 2056, 2072 and the nozzle assembly 2014. The light pipe preferably is in the shape of a circle that abuts the circumference of the mesh plate 2056, 2072. An illumination device such as LEDs 3006 is mounted on either side of the container 2024 as shown in FIG. 41a. The light of the LEDs 3006 is guided through the light pipe 3004 and would be most visible near the opening from which the mist is dispersed particularly if a diffraction texture is applied to the surface of the light pipe. The end result is a ring of light is emitted which makes it easier to align the device. For example, the eye to which the fluid was to applied could be centered within the ring of light before application of the fluid. Or, for self-administration, the light ring acts to orient and fixate the eye in the direction of the plume emission.

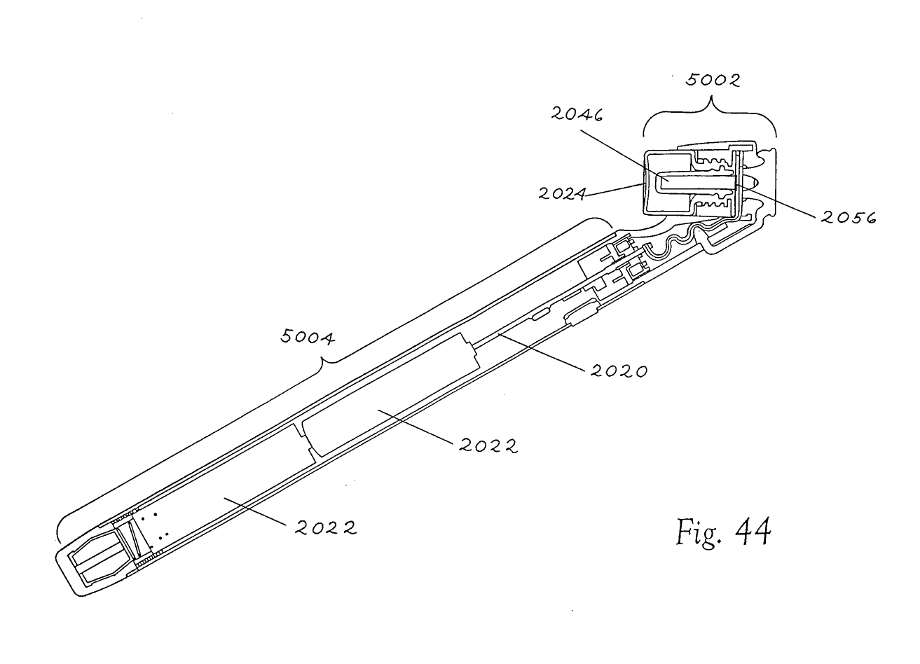

Another example of a possible modification of the embodiments of FIGS. 1-41b is shown in FIGS. 42-51. In particular, the various internal components described in FIGS. 1-41b and below can be contained in the design shown in FIGS. 42-51. In one variation, the design includes handle portion 5004, head portion 5002, container 2024 partially contained within head portion 5002 and which threadedly engages an internal thread of head portion 5002, wick 2046 and mesh plate 2056 as shown in FIG. 44. The variation of FIG. 44 operates in a manner similar to that described with respect to the embodiments of FIGS. 28-41b.

Other features of interest are that the wick system enables the device to be used in any orientation. The wick contains a substantial amount of the fluid in the medule, up to 100% if desired. The fluid flow to the discharge plate is regulated by capillary force. While not wishing to be constrained by theory, it is believed that as the fluid is delivered to the discharge plate, air is admitted to the reservoir through the same wick. Depending on the pore sizes in the wick and whether the wick is coated with ionic silver, the wick can act as a filter to prevent introduction of bacteria. Without a wick, the device shows markedly different dispensing characteristics when the discharge is oriented down, sideways or up. More fluid is atomized when oriented down. Less is atomized when oriented sideways and even less when oriented up. Atomization in the sideways orientation is dependent on the level of fluid in the reservoir. When low, only the amount of fluid clinging to the proximal side of the discharge plate is available for dispensing. So, the wick acts to regulate the amount of fluid present on the proximal side of the discharge plate. Further, the wick acts as a buffer, enabling multiple dispense cycles without reorienting the device. Without the wick, the device would need to be shaken or re-oriented between each dose to ensure that the discharge plate was wetted. Finally, the wick substantially improves the reliability of the device.

Another possibility is to use a silver ion coating, especially for the discharge plate, so as to substantially reduce the formation of biofilms by inhibiting bacterial growth. Additionally, it is believed that silver ion coating of the wick passages may be beneficial by providing an anti-bacterial effect. This may enable the storage and delivery of fluids without preservatives.

We claim:

1. An ophthalmic fluid atomizer configured to safely deliver an ophthalmic fluid to an eye, the ophthalmic fluid atomizer comprising:
a body having a proximal end and a distal end;
a container connected to the body, wherein the container contains an ophthalmic fluid disposed therein;
a discharge plate disposed at the distal end, wherein the discharge plate includes a plurality of openings extending therethrough;
a wick extending from the container to the discharge plate and transmitting the ophthalmic fluid from the container to the discharge plate via capillary action, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye;
a processor that controls vibration of the discharge plate which causes an aerosol mist to form;
an activation switch operatively coupled to the processor to activate the processor.

2. The ophthalmic fluid atomizer according to claim 1, wherein the atomizer discharges the ophthalmic fluid having a velocity of between approximately 4 and 30 centimeters per second.

3. The ophthalmic fluid atomizer according to claim 1, wherein the atomizer discharges the ophthalmic fluid between approximately 2 and 10 microliters per second.

4. The ophthalmic fluid atomizer according to claim 1, wherein the atomizer operates for approximately 0.5 to 5 seconds upon activation of the activation switch.

5. The ophthalmic fluid atomizer according to claim 1, wherein the atomizer discharges the ophthalmic fluid having average particle sizes between approximately 0.5 and 10 microns in diameter.

6. The ophthalmic fluid atomizer according to claim 1, wherein the activation switch is configured to generate a signal to the processor that leads to controlling transmission of the ophthalmic fluid for a predetermined period of time, the predetermined period of time having a duration which is independent of operation of the activation switch.

7. The ophthalmic fluid atomizer according to claim 1, wherein the container is removable.

8. The ophthalmic fluid atomizer according to claim 1, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

9. The ophthalmic fluid atomizer according to claim 8, wherein the glaucoma medications are selected from the group consisting of prestaglandins, beta blockers, alpha adrenergic agents, carbonic anhydrase inhibitors, and miotics.

10. The ophthalmic fluid atomizer according to claim 1, wherein the plume of ophthalmic fluid has a volume that can entirely be retained by the eye.

11. The ophthalmic fluid atomizer according to claim 1, wherein the plume of ophthalmic fluid has a volume of at most approximately 5 micro liters.

12. The ophthalmic fluid atomizer of claim 1, wherein the plume of ophthalmic fluid contains an amount of ophthalmic medicine and the momentum of the plume is such that substantially all of the amount of ophthalmic medicine is received and retained by the human eye.

13. The ophthalmic fluid atomizer of claim 1, wherein the mist contains an amount of ophthalmic medicine and the momentum of the mist is such that substantially all of the amount of ophthalmic medicine is received and retained by the human eye 14.

14. The ophthalmic fluid atomizer according to claim 1, wherein the atomizer discharges the ophthalmic fluid having a velocity of between approximately 4 and 30 centimeters per second.

15. The ophthalmic fluid atomizer according to claim 1, further comprising a cap that encloses the discharge plate and wherein at least a portion of the cap is transparent; and wherein the container comprises indicia that identifies the ophthalmic fluid, the indicia being visible to an observer via the at least a portion of the cap that is transparent.

16. The ophthalmic fluid atomizer according to claim 1, further comprising a glowing ring surrounding the discharge plate.

17. The ophthalmic fluid atomizer according to claim 15, further comprising a glowing ring surrounding the discharge plate.

18. The ophthalmic fluid atomizer according to claim 1, wherein the processor controls vibration of the discharge plate so that a sequence of pulses of mist is delivered to the eye.

19. The ophthalmic fluid atomizer according to claim 1, wherein the processor applies pulse width modulation to control vibration of the discharge plate so as to control a flow rate of the mist.

20. The ophthalmic fluid atomizer according to claim 1, wherein the wick is coated with a coating selected from the group consisting of ionic silver and PTFE.

21. The ophthalmic fluid atomizer according to claim 1, wherein the discharge plate is coated with a coating selected from the group consisting of ionic silver and PTFE.

22. The ophthalmic fluid atomizer according to claim 1, further comprising a diaphragm positioned between the wick and the discharge plate, the diaphragm being in an open position when the mist is generated and in a closed position otherwise.

23. The ophthalmic fluid atomizer according to claim 22, wherein the wick or discharge plate automatically move relative to one another when the diaphragm moves from the open position to the closed position.

24. The ophthalmic fluid atomizer according to claim 21, further comprising a diaphragm positioned between the wick and the discharge plate, the diaphragm being in an open position when the mist is generated and in a closed position otherwise.

25. The ophthalmic fluid atomizer according to claim 24, wherein the wick or discharge plate automatically move relative to one another when the diaphragm moves from the open position to the closed position.

26. The ophthalmic fluid atomizer according to claim 25, wherein UV light, low pH formulations and chemically preserved solutions are applied to the discharge plate in order to prevent microbial contamination of the mist.

27. The ophthalmic fluid atomizer according to claim 1, wherein UV light is applied to the discharge plate in order to prevent microbial contamination of the mist.

28. The ophthalmic fluid atomizer according to claim 1, further comprising a diaphragm positioned so that the discharge plate lies between the diaphragm and the wick, the diaphragm being in an open position when the mist is generated and in a closed position otherwise.

29. An ophthalmic fluid atomizer configured to safely deliver an ophthalmic fluid to an eye, the ophthalmic fluid atomizer comprising:
a body having a proximal end and a distal end;
a medule system releasably connected to the body, the medule system comprising:
a container containing an ophthalmic fluid disposed therein and defining a first opening; and
a wick that is inserted into the opening and extending into the container so as to contact the ophthalmic fluid.
a discharge plate disposed at the distal end, wherein the discharge plate includes a plurality of openings extending therethrough;
wherein the wick extends from the container to the discharge plate and transmits the ophthalmic fluid from the container to the discharge plate via capillary action, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye.

30. The ophthalmic fluid atomizer of claim 29, wherein the medule system, comprises a plug inserted into the opening, the plug defining a second opening through which the wick is inserted.

31. The medule system of claim 30, wherein the container comprises a neck portion that comprises a first thread, wherein the plug engages the neck via an interference fit.

32. The medule system of claim 30, wherein the opening is aligned with the second opening.

33. The medule of claim 29, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

34. The medule of claim 33, wherein the glaucoma medications are selected from the group consisting of prestaglandins, beta blockers, alpha adrenergic agents, carbonic anhydrase inhibitors, and miotics.

35. A medule system, comprising:
a container containing an ophthalmic fluid disposed therein and defining a first opening;
a plug inserted into the first opening, the plug defining a second opening; and
a wick that is inserted into the first opening and the second opening and extending into the container so as to contact the ophthalmic fluid.

36. The medule system of claim 35, wherein the container comprises a neck portion that comprises a first thread, wherein the plug engages the neck via an interference fit.

37. The medule system of claim 35, wherein the first opening is aligned with the second opening.

38. The medule of claim 35, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

39. The medule of claim 38, wherein the glaucoma medications are selected from the group consisting of prestaglandins, beta blockers, alpha adrenergic agents, carbonic anhydrase inhibitors, and miotics.

40. An ophthalmic fluid atomizer configured to safely deliver an ophthalmic fluid to an eye, the ophthalmic fluid atomizer comprising:
a body having a proximal end and a distal end;
a container connected to the body, wherein the container contains an ophthalmic fluid disposed therein;
a discharge plate disposed at the distal end, wherein the discharge plate includes a plurality of openings extending therethrough;
a prime mover extending from the container to the discharge plate and transmitting the ophthalmic fluid from the container to the discharge plate, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye;
a glowing alignment device surrounding the discharge plate;
a processor that controls vibration of the discharge plate which cause an aerosol mist to form;
an activation switch operatively coupled to the processor to activate the processor.

41. The ophthalmic fluid atomizer according to claim 40, wherein the alignment device comprises a light pipe and an illumination device for dispersing light into the light pipe.

42. The ophthalmic fluid atomizer according to claim 41, wherein the light pipe is symmetrically positioned with respect to the prime mover.

43. The ophthalmic fluid atomizer according to claim 40, wherein the prime mover comprises a wick.

44. The ophthalmic fluid atomizer according to claim 40, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

45. The ophthalmic fluid atomizer according to claim 44, wherein the glaucoma medications are selected from the group consisting of prestaglandins, beta blockers, alpha adrenergic agents, carbonic anhydrase inhibitors, and miotics.

46. An ophthalmic fluid atomizer configured to safely deliver an ophthalmic fluid to an eye, the ophthalmic fluid atomizer comprising:
a body having a proximal end and a distal end;
a container connected to the body, wherein the container contains an ophthalmic fluid disposed therein and the container comprises indicia indicative of the identity of the ophthalmic fluid;
a discharge plate disposed at the distal end, wherein the discharge plate includes a plurality of openings extending therethrough;
a prime mover extending from the container to the discharge plate and transmitting the ophthalmic fluid from the container to the discharge plate, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye;
a cap that covers the discharge plate and the prime mover, wherein a portion of the cap is transparent and positioned so that a user can see the indicia through the portion of the cap.

47. The ophthalmic fluid atomizer according to claim 46, further comprising:
a processor that controls vibration of the discharge plate which cause an aerosol mist to form; and
an activation switch operatively coupled to the processor to activate the processor.

48. The ophthalmic fluid atomizer according to claim 46, wherein the cap is totally transparent.

49. The ophthalmic fluid atomizer according to claim 46, wherein the prime mover comprises a wick.

50. The ophthalmic fluid atomizer according to claim 46, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

51. The ophthalmic fluid atomizer according to claim 50, wherein the glaucoma medications are selected from the group consisting of prestaglandins, beta blockers, alpha adrenergic agents, carbonic anhydrase inhibitors, and miotics.

52. An ophthalmic fluid atomizer configured to safely deliver an ophthalmic fluid to an eye, the ophthalmic fluid atomizer comprising:
a body having a proximal end and a distal end;
a container connected to the body, wherein the container contains an ophthalmic fluid disposed therein;
a discharge plate disposed at the distal end, wherein the discharge plate includes a plurality of openings extending therethrough;
a prime mover extending from the container to the discharge plate and transmitting the ophthalmic fluid from the container to the discharge plate, wherein transmission of the ophthalmic fluid across the discharge plate generates a plume of ophthalmic fluid along a direction directly toward the eye, wherein the plume of ophthalmic fluid travels unassisted from the discharge plate to the eye and at the eye has a momentum subliminal to at least one of an ocular blink reflex and a lacrimation reflex of the eye;
a closure element that moves from a first position to a second position, wherein the closure element at the first position prevents the plume from reaching the eye and the closure element at the second position allows the plume to reach the eye.

53. The ophthalmic fluid atomizer according to claim 52, wherein the prime mover or discharge plate automatically move relative to one another when the closure element moves from the first position to the second position.

54. The ophthalmic fluid atomizer according to claim 52, wherein the discharge plate is positioned between the prime mover and the closure element.

55. The ophthalmic fluid atomizer according to claim 54, wherein the closure element is a pivoting cap.

56. The ophthalmic fluid atomizer according to claim 52, wherein the closure element is positioned between the prime mover and the discharge plate.

57. The ophthalmic fluid atomizer according to claim 56, wherein the closure element is an iris diaphragm.

58. The ophthalmic fluid atomizer according to claim 53, wherein the discharge plate is positioned between the prime mover and the closure element.

59. The ophthalmic fluid atomizer according to claim 58, wherein the closure element is a pivoting cap.

60. The ophthalmic fluid atomizer according to claim 52, wherein the prime mover comprises a wick.

61. The ophthalmic fluid atomizer according to claim 52, wherein the ophthalmic fluid is selected from the group consisting of mydriatics/cycloplegics, anesthetics, flourescein, flourescein/anesthetic combinations, mydriatic reversal agents, ophthalmic decongestants, ophthalmic lubricants, and glaucoma medications.

62. The ophthalmic fluid atomizer according to claim 52, wherein the glaucoma medications are selected from the group consisting of prestaglandins, beta blockers, alpha adrenergic agents, carbonic anhydrase inhibitors, and miotics.
